(19) United States
(12) Patent Application Publication
(10) Pub. No.: US 2024/0202922 A1
(43) Pub. Date: Jun. 20, 2024

(54) PLANT MANAGEMENT SYSTEM

(71) Applicant: CROPSY TECHNOLOGIES LIMITED, Avondale, Auckland (NZ)

(72) Inventors: Leila DELJKOVIC, New Lynn, Auckland (NZ); Ali Sarmad Saieb AL-OMARI, New Lynn, Auckland (NZ); Winston Ronald SU, Mt Eden, Auckland (NZ); Rory Douglas BUCHANAN, New Lynn, Auckland (NZ)

(21) Appl. No.: 18/555,702

(22) PCT Filed: Apr. 14, 2022
(86) PCT No.: PCT/NZ2022/050042

§ 371 Date: Oct. 16, 2023

(30) Foreign Application Priority Data
Apr. 15, 2021 (NZ) 775098

(51) Int. Cl. G06T 7/00; G06T 7/593; G06V 10/141; G06V 10/60; G06V 20/10; G06V 20/56

(52) U.S. Cl. CPC G06T 7/0012; G06T 7/593; G06V 10/141; G06V 10/60; G06V 20/188; G06V 20/56; G06T 2207/10012; G06T 2207/10028; G06T 2207/30188; G06T 2207/30252

(57) ABSTRACT

A method comprising: activating an illumination source to produce illuminating light: polarising the illuminating light in a first polarisation axis; illuminating at least part of a thin canopy plant crop with the polarised illuminating light to produce reflected illuminating light; polarising the reflected illuminating light in a second polarisation axis transverse to the first polarisation axis to produce cross-polarised reflected illuminating light; capturing an image of at least part of the thin canopy plant crop using the cross-polarised reflected illuminating light; and analysing the captured image to determine a condition of the thin canopy plant crop. Also an image capture system and a vehicle mounted image capture system.

### FIELD

This invention relates to a plant management system.

### BACKGROUND

Various types of machine vision systems exist. In relation to management of plant health, these may be used in detecting diseases or pests, or determining other characteristics or features of plants in short crops.

Machine vision use in crops is not without problems. For example: limited annotated datasets, symptom representation, covariate shift, image background, image capture conditions, symptom segmentation, symptom variations, simultaneous disorders solved, disorders with similar symptoms and/or specular lighting.

One of the more problematic image capture conditions is ambient illumination, in particular the variable effect of the sun on the image data set. The variable effect of the sun can prove challenging to overcome in a commercial setting. Additionally, deploying a machine vision system to manage plants in a commercial setting can be very challenging if the system is not easily scalable to the entire crop, or not able to be used frequently enough to bear commercial value to the growing operation.

Machine vision systems in use may also lack the ability to image details and surface features of plants from a close distance.

### SUMMARY

According to one example embodiment there is provided a method according to claim 1, or a system according to any one of claims 16 and 23.

Embodiments may be implemented according to any one of the dependent claims.

It is acknowledged that the terms “comprise”, “comprises” and “comprising” may, under varying jurisdictions, be attributed with either an exclusive or an inclusive meaning. For the purpose of this specification, and unless otherwise noted, these terms are intended to have an inclusive meaning—i.e., they will be taken to mean an inclusion of the listed components which the use directly references, and possibly also of other non-specified components or elements.

Reference to any document in this specification does not constitute an admission that it is prior art, validly combinable with other documents or that it forms part of the common general knowledge.

### BRIEF DESCRIPTION OF THE DRAWINGS

The accompanying drawings which are incorporated in and constitute part of the specification, illustrate embodiments of the invention and, together with the general description of the invention given above, and the detailed description of embodiments given below, serve to explain the principles of the invention, in which:

FIG. 1 is a front view of a tractor including an image capture system according to an example embodiment;

FIG. 2 is a plan view of the tractor in FIG. 1 including an automated spraying system and a blower;

FIG. 3A is a perspective view of the image capture device in FIG. 1 without a spray shield;

FIG. 3B is a perspective view of the image capture device in FIG. 1 with a spray shield;

FIG. 4 is a schematic depiction of image capture system according to an example embodiment;

FIG. 5 is a rear view of a tractor showing an alternate capture device connection structure;

FIG. 6A is a rear view of the tractor in FIG. 2 with the addition of alternative leaf blowers;

FIGS. 6B to 6E are perspective views of four possible implementations of the leaf blowers in FIG. 6A;

FIG. 7 is a schematic diagram of the sprayer system in FIG. 2;

FIG. 8 is a block diagram of the detection controller in FIG. 7;

FIG. 9 is a block diagram of the base and cloud controllers;

FIG. 10 is a front view of the tractor shown in FIG. 2, with an automated spraying system spanning multiple rows;

FIG. 11 is a plan view of the tractor in FIG. 10;

FIGS. 12A to 12G are a flowchart of the software processes executed in the detection controller to handle real-time detection and data storage for later viewing;

FIGS. 13A to 13B are a flowchart of the software processes executed in the base station and cloud to aid with geo-tagging and storage and display of detection and treatment information;

FIG. 14 is a use-case diagram specifying the setup steps of the system;

FIG. 15 is a use-case diagram specifying the different user interactions with the system during detection and automated treatment dispensing;

FIG. 16 is a use-case diagram specifying user interaction following a detection/treatment session;

FIG. 17 is a circuit diagram of a filter circuit for the LED control signal

FIG. 18 is a front view of an imaging device according to one example embodiment;

FIG. 19 is a graph of operation of a non-cooled LED;

FIG. 20 is a graph of operation of a cooled LED;

FIG. 21 is another graph of operation of a cooled LED;

FIG. 22 is a front view of an arrangement of an imaging device; and

FIG. 23 is a side view of the arrangement of FIG. 22.

FIG. 24 is a safety discharge circuit according to one embodiment;

FIG. 25 is a perspective view of a leaf blower according to one example embodiment;

FIG. 26 is a side view of the leaf blower of FIG. 25;

FIG. 27 is front view of the leaf blower of FIG. 25;

FIG. 28 is a rear view of the leaf blower of FIG. 25;

FIG. 29 is a block diagram of the leaf blower hardware according to one example embodiment;

FIG. 30 is a flow chart of a leaf blower control algorithm according to one example embodiment;

FIG. 31 is a rear view of a tractor showing an alternative integrated image capture and controller for tall crops;

FIG. 32 is a rear view of a tractor showing an alternative upwards facing image capture for thin canopy crops;

FIG. 33 is a rear view of a tractor showing an alternative integrated image capture and controller for thin canopy crops;

FIG. 34 is a perspective view of the image capture device in FIG. 32 without a spray shield;

FIG. 35 is a perspective view of the image capture device in FIG. 32 with a spray shield;

FIG. 36 is a view of a first example swivel mount in a first state;

FIG. 37 is a view of the swivel mount of FIG. 36 in a second state;

FIG. 38 is a view of a second example mount;

FIG. 39 is a view of a third example mount;

FIG. 40 is a view of a fourth example mount; and

FIGS. 41A and 41B are a top and side view of an example passive cooling device; and

### DETAILED DESCRIPTION

In order to reduce the effect of variability of sun illumination on short crop machine vision, one option is to use a sun shield. In that case the cameras might be generally downward facing, for example, seeing a bird's-eye-view. The cameras and spraying system may be integrated into a tractor attachment. However, employing a sun shield for plant species, other than short crops, may prove commercially challenging in some applications. In other words, an alternative solution may be required in some applications.

Depending on the application, embodiments may seek to improve image capture of “tall crops” or “tall plants”, as opposed to “short crops”. Depending on the application, the distinction between short and tall may differ. Some indicia of a tall crop may include:
Mostly woody trees or vines which are planted in the ground in distinct rows;
Narrow in width as opposed to full and bushy, such that the camera may capture sufficiently large areas of the foliage from either side of the row while viewing the foliage from the side, or that any fruit or produce from the plant could not effectively be seen from above;
Grown vertically with supports or training systems such as posts, trellises, wires, ties, bindings, etc. Short or bushy crops generally aren't “trained”.
Grown using training systems that create a flat, narrow canopy; and/or
Applications where the camera must be facing sideways generally indicate that a crop is too tall to be covered easily, otherwise a camera could look down on the crop, and it could be covered with a sun shield.

Depending on the application, embodiments may alternatively seek to improve image capture of “thin canopy crops” or “thin canopy plants”, as opposed to “short crops”. The canopy of a plant is the above-ground portion of the plant which may form from branches, stems, or other structures that extend from the trunk of the plant. Depending on the application, the distinction between short and thin canopy may differ. Some indicia of a thin canopy crop may include:
Mostly woody trees or vines which are planted in the ground in distinct rows;
The rows may be broad in width and the canopy may be trained to grow in a flat and thin fashion which is elevated from the ground, such that the camera may capture sufficiently large areas of the foliage or produce from the underside of the canopy while viewing the foliage from the underside, or that any fruit or produce from the plant could not effectively be seen from above;
Grown vertically from the base or trunk of the plant and trained to form a flat, thin canopy. This canopy may be formed in a vertical fashion, or horizontal fashion, or angled fashion where the foliage from two rows may meet between a row to create a canopy which is uniform in appearance. The training of the plant may be achieved with supports or training systems such as posts, trellises, wires, ties, bindings, etc. Short, shrub-like, or full bushy crops generally aren't “trained”.
Grown using training systems that create a flat, thin canopy; and/or
Applications where the camera must be facing upwards, or angled upward, or sideways generally indicate that a crop is too high or inaccessible to be covered easily, otherwise a camera could look down on the crop, and it could be covered with a sun shield.

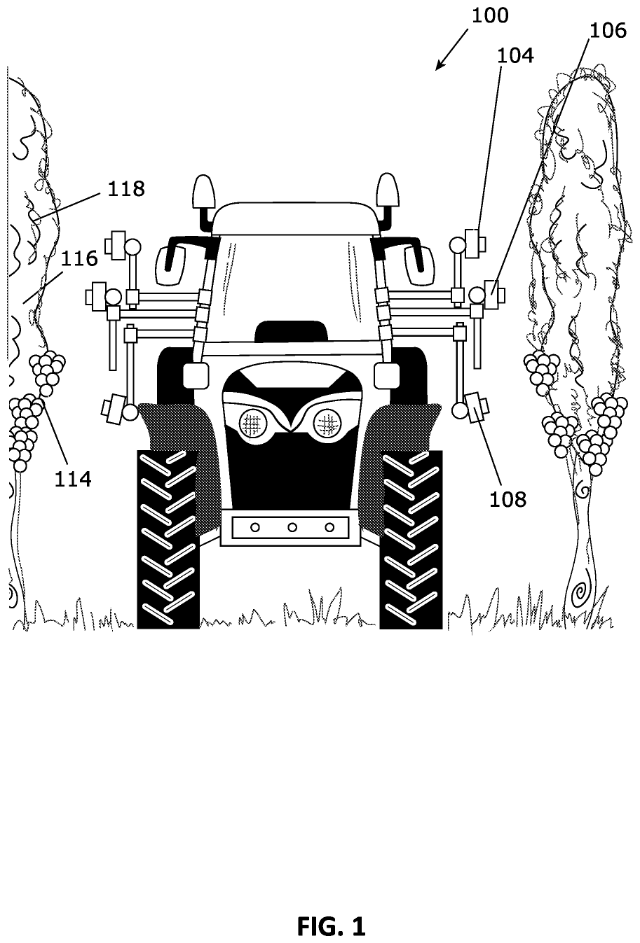

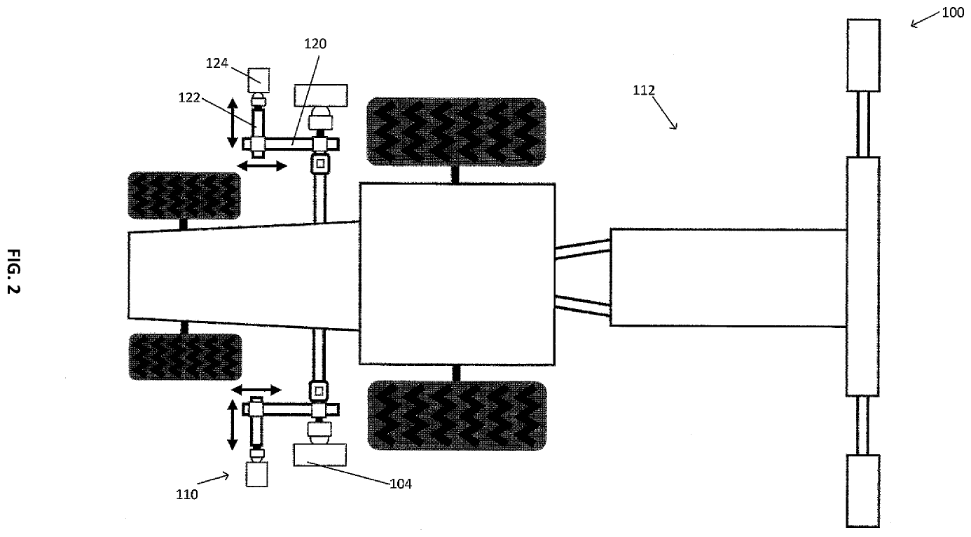

FIGS. 1 and 2 illustrate a vehicle mounted image capture and analysis system 100 according to an example embodiment. The main components include an illumination source and multiple image capture devices 104, 106, 108. It may include a blower 110 to displace the foliage and/or produce and may also include an automated spray system 112 to spray based on the results of the image capture, or other output application modules.

Depending on the application, vehicle mounted may mean that the vehicle itself is multipurpose, such as a tractor, or spraying vehicle, harvesting vehicle, or all-terrain vehicle (ATV), and the image capture devices can be detached if the vehicle needs to be used for other purposes. This is as opposed to a purpose designed vehicle or robot just for capturing images or analysing images. However, the camera may also be attached to a vehicle or robot designed for capturing images or analysing images or for other purposes. These vehicles or robots may also be autonomous.

### Image Capture

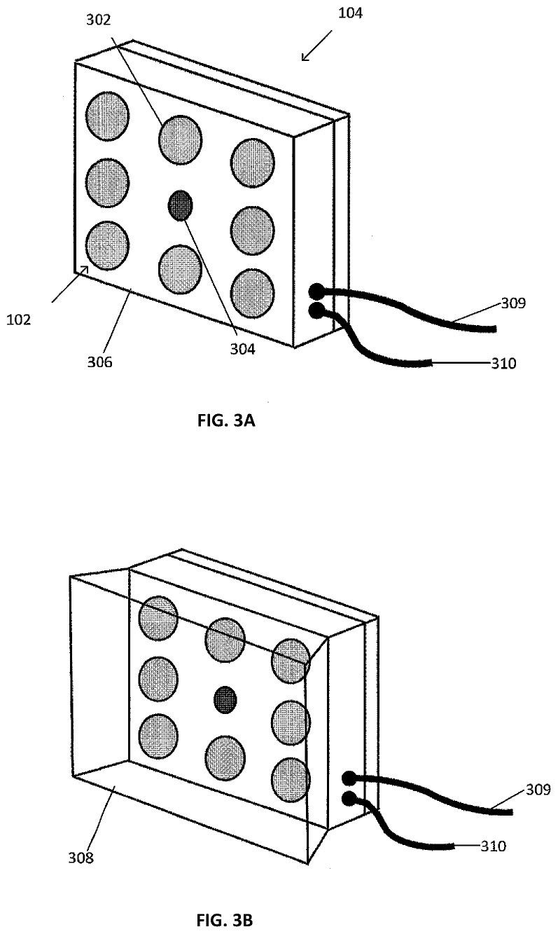

An example image capture device 104, 106, 108 is shown in FIGS. 3A and 3B. An illumination source 102, may take the form of 8 or more LEDs 302, and the LEDs 302 may surround a CMOS sensor or sensors 304 in the centre of a housing 306. Surrounding the imaging component with the lighting components improves the evenness and consistent distribution of light in the captured image. This is also a compact way of achieving evenness and consistent distribution. Generally speaking, the closer the origin of light is to the imaging device, the closer the field of the view of the imaging device will be to the illumination field of the lighting device. Consequently, the more homogenous and consistent the lighting in the collected images will be, regardless of the distance at which the plant is imaged.

As shown in FIG. 3B, housing 306 may include a shield 308 to prevent water droplets from rain or sprays adhering to the cover of the housing, preventing accumulation of dust and fine debris, and preventing splashes of mud or water from landing on the cover of the housing, all of which may affect the visibility of the CMOS sensor 304. The cover itself may be of high optical transmission (96% or greater), for example optical acrylic or optical glass, and may have scratch-resistant or anti-reflective coatings. Additionally, the cover may be flush with the lens inside the enclosure such that light does not ‘leak’ into the lens from inside the enclosure. Foam padding may be used to achieve this and provide some damping to prevent damage to the lens or camera from vibrations or shock.

The shield 308 may be transparent to prevent light from LEDs 302 being blocked, or it may be opaque to act as a lens hood depending on the view of the CMOS sensor 304, as required by the application. An opaque cover would also help to shield the vehicle operator from the bright flashes which could be distracting or cause eye discomfort. Additionally, the opaque cover may be white, somewhat reflective or may be coated with a reflective material to redirect some light towards the CMOS sensor's field of view. This may additionally improve the image brightness. The image capture device 104 is connected to a power supply via line 309 and to a single-board computer or processor via data line 310.

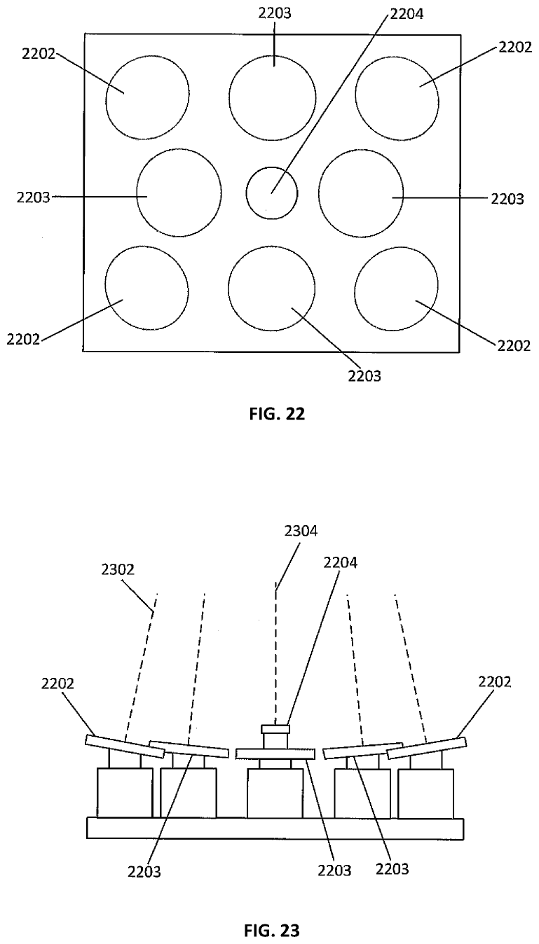

FIGS. 22 and 23 show one exemplary arrangement of the illumination sources, which can be the LEDs as discussed above. They are arranged around the imaging device 2204. As best seen in FIG. 23, the illumination sources are mounted at an angle so that their respective illumination axes 2302 are tilted, either towards the imaging axis 2304 of the imaging device 2204, or to converge at two or more points surrounding the imaging device 2204. In one instance, those LEDs that are further out from the imaging device 2204 can be tilted by a greater amount so that the light from all imaging devices converges within the field of view of the imaging device 2204. In another instance, those LEDs on one side of the imaging device can be tilted to converge slightly to the left of the imaging axis and those LEDs on the other side of the imaging device and converge slightly to the right of the imaging axis. This may result in a relatively higher level of illumination at one point of the image, relative to the generally even level of illumination across the remainder of the image.

The angle at which they are mounted may be adjusted to change the distance at which the light converges. For example, the outer lights 2202 are angled at 11 degrees, and the inner ones 2203 are at 6 degrees, based on the subject being 0.4 m to 0.8 m away, and the specific layout of the lights. An option to account for real-time variation in canopy depth and density may use a single or a combination of distance measurement sensors including but not limited to depth cameras, Lidar, infrared or light reflective sensors, or others to adjust the angles of the illumination sources to maintain the convergence point at the average depth of the canopy and maximise light concentration and image homogeneity. Mechanical actuation devices including but not limited to servo motors, linear actuators may be used directly or indirectly through an actuation system to control the angle of the lights.

This arrangement increases the effective light intensity in the imaged area without the need to add extra light sources or increase the applied voltage to the light sources. Because the light is concentrated in the field of view of the imaging device, less light is ‘wasted’ outside of the field of view.

A laser can also be provided with the imaging device to highlight the field of view of the imaging device. A diffraction grating can be provided to split the laser and shape it to match the field of view of the imaging device. When more than one imaging device is used, the projected laser light from all of the imaging devices can be aligned to align all of the fields of the view of the imaging devices.

The CMOS sensor 304 is a colour sensor, and it uses a global shutter. An optical band-reject filter may be used to block unwanted frequencies of light, for example an infra-red cut filter.

Using a colour sensor instead of a monochromatic sensor may help to detect and measure a wider range of different pests, diseases, and plant features. Colour information may also be used to identify other issues such as nutritional deficiencies, water stress, canopy “greenness”, canopy density or vigour, leaf-area index, missing vines, estimating fruit ripeness, aid in counting flowers, buds, and fruit for yield estimation, or aid in counting trunks, canes, cordons, and shoots, or aid in counting posts, depending on the requirements of the application. For example, the image could be segmented based on what's green and what isn't, and calculating the percentage of green to measure canopy density. Counting trunks is useful to track how many plants there are in the crop, as some plants often need to be uprooted because of incurable diseases. Missing, dying or dead plants could also be found using the canopy density method in conjunction with counting trunks. It could also be a basis for leaf-area index (again, finding green areas which happen to be leaf shaped). Counting posts and mapping out their locations is useful as they're often used as landmarks in the crop to tell workers where they need to go.

The global shutter prevents any distortions, for example, motion blur, in the images while the vehicle is moving. In a vineyard, for example, tractors will typically go 8 to 12 km/h, and ATVs will go up to 30 km/h.

To reduce the effect of ambient illumination and specular reflection, various approaches may be taken, according to the requirements of the application. First a very low exposure time may be used for the CMOS sensor 304. Depending on the environmental variables, and the sensor used, this may be similar to high speed photography, and in one example the exposure may be between 30-200 us.

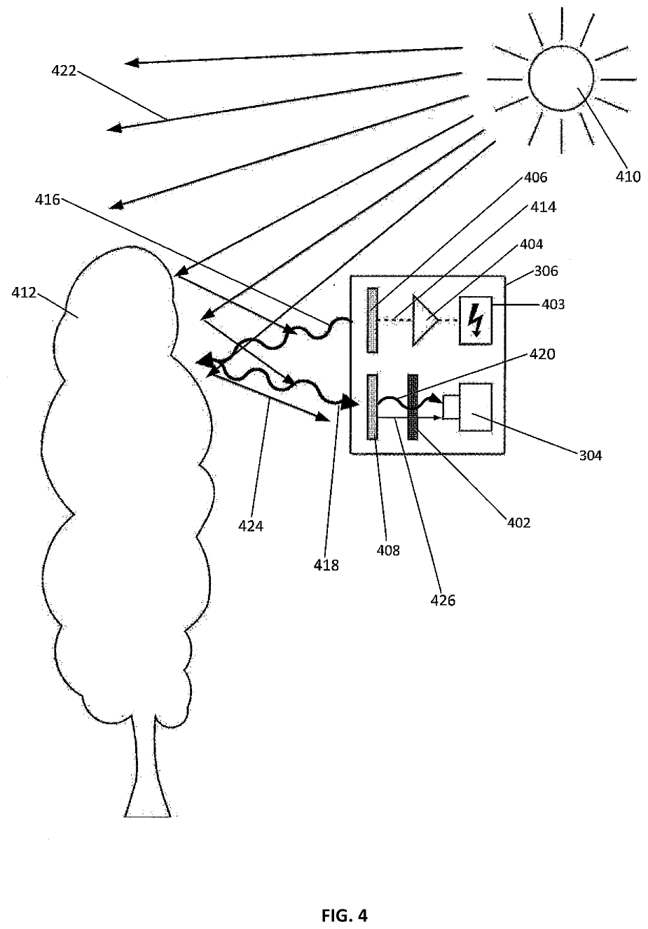

Secondly as shown in FIG. 4, the image capture device 104 may incorporate a neutral density filter 402 in front of the sensor 304 to darken the scene further. The neutral density filter 402 may for example be a single filter or multiple filters stacked on top of each other to produce a collective darkening effect corresponding to an optical density between 0.046 to 3.0. The neutral density filter 402 may be designed to darken the image and affect all colours equally such that the ratios between darkened colours are preserved and the image colour integrity is maintained. The neutral density filter 402 may be designed to also absorb or reflect wavelengths outside the visible spectrum such as infrared or UV light, e.g. below 380 nm or above 740 nm.

Thirdly the lens aperture of the CMOS sensor 304 may be adjusted to darken the scene. For example, the aperture may be adjusted to f/16 between f/8 and f/16, (though beyond f/16 could also make the image even darker) with a focal length of 6 mm to ensure adequate depth of field and that the image is in focus, and (if necessary) to minimise barrel distortion.

Fourthly as shown in FIG. 4 cross polarisation may be used to reduce reflection artefacts from the leaves or other parts of the plant. Reflection artefacts can make it difficult or impossible to image surface features of the plant. While simply imaging the overall shape of fruit or other plant parts may be enough to roughly estimate yield, canopy cover, leaf-area index etc. in some situations, some analyses require analysis of the surface of the plant. For example, pest and disease detection may use segmentation of the image of leaf surfaces to identify blemishes caused by fungal bodies, signs of pests, or pests themselves. Reflections obfuscate these surface features making it unclear or impossible to view them.

In the example of FIG. 4, a plant 412 is being imaged during the day, with ambient light 422 being produced by the sun 410. The illumination source produces illuminating light 414 for illuminating the plant 412. The illuminating light is passed through a first polarising filter 406 to produce polarised illuminating light 416. The polarised illuminating light is reflected off the plant 412 to produce reflected polarised illuminating light 418. The reflected polarised illuminating light 418 is passed through a second polarising filter 408 that has a polarisation axis transverse to that of the first polarising filter 406. This cross-polarises the light 418 to produce cross-polarised reflected illuminating light 420 with significantly reduced specular reflections. This cross-polarised light 420 is captured by the image capture device for imaging the plant 412.

Ambient light 422 is also reflected from the plant 412 to produce reflected ambient light 424. This is inevitable in a situation where the imaged plant cannot be shaded from ambient light, which is typically the case in the field. The reflected ambient light 424 will also pass through the second polarising filter 408 to produce polarised reflected ambient light 426 which will also enter the image capture device along with the cross-polarised light 420.

The illumination source produces high-intensity light so that the cross-polarised light 420 received by the imaging device is of a greater intensity than the polarised reflected ambient light 426. This reduces the presence of reflections from the ambient light (which is not cross-polarised) in the captured image. The illumination source can be bright enough that the intensity of the cross-polarised light is 2, 5, 10 or more than 10 times greater than that of the polarised reflected ambient light 426. In the case that ambient light is provided by the sun 410, this means that the illumination source's light needs to overpower the sun's light in the captured image by this amount. As discussed below, this can be achieved using overdriven LEDs, although other suitable illumination sources such as halogen bulbs or high intensity sources could be used in some applications.

Using a polarised active illumination source to illuminate the plant avoids the need to polarise ambient light such as sunlight. This makes the system more convenient and suitable for use in the field because light from a lighting device like an LED can easily be polarised by a small polariser carried around with the device, whereas polarising ambient light requires large and unwieldy polarisers that are not easily transportable. Active lighting also minimises shadows in the image and more evenly illuminates the plant to expose the features and colours of the plant parts such as leaves, fruit and stems—particularly in outdoor conditions.

As noted above, using high-intensity illumination sources that overpower ambient light sources avoids the need to shade the plant from ambient light during imaging. When the illumination sources are sufficiently intense to overpower the sun, this enables imaging to be performed during the day on unshaded plants. This is much more convenient than only imaging at night or in other low-light conditions. The greater the degree to which the illumination source overpowers the sun, the less the reflections from the sunlight affect the captured image.

The system can be used to analyse the surface of plants outdoors in a range of conditions, which makes it much more suitable for large-scale applications in the field. Improved surface imaging and analysis improves the ability to detect or segment bunches of fruit, shoots, berries and buds to improve yield estimates. It also allows for the detection of pests and diseases, nutritional deficiencies, water stress, chemical residues (which indicate spray efficiency), fruit colour (which indicates ripeness and can be used to sort fruit for different markets according to its colour profile, for example), leaf-area index, canopy density, posts (this help growers identify which bay and row an infection is in, and which may be used instead of or in addition to GNSS location), broken posts which need to be replaced, plant trunks (to keep track of missing plants), and untreated cuts. When the plants are pruned, a brightly coloured seal may be painted onto the open cuts to prevent diseases from infecting the plant. If so, using a colour imaging sensor also makes it easy to spot if a cut has been treated or not.

Loss of light through a polariser should be taken into account when designing the system. A dichroic linear polarising film, for example, will typically only transmit up to about 42% of incident light. This means that with two of these polarisers, the total transmitted light will be up to about 0.42*0.42=0.18 or 1/5.6. The illumination source should therefore be at least 5.6 times as intense as it would otherwise need to be without the polarisers.

Each of the 8 or more LEDs 302 may include a white LED 403, a LED lens 404 in front of the LED 403, and a 0° polarising filter 406 in front of the lens 404. The CMOS sensor 304 may include a 90° polarising filter 408 mounted in front of a neutral density filter 402. The polarisers need not be at exactly 90° to each other to be effective, although they will be most effective when closest to 90° to each other.

The LED lens mounts, LED lenses, heatsinks may all be mounted to a PCB board with a hole in the centre for the CMOS sensor. The PCB may be fixed to the housing 306 with screws and may take up most of the space in the housing 306. A water and dust resistance rating of IP65 for the housing will be sufficient to resist dust, rain, humidity, or sprays. The housing 306 may include a waterproof vent to allow humidity to escape and prevent condensation, such as a GORE™ Vent. The housing 306 may be a type of plastic or reinforced plastic that is UV stabilised, and not deform when out in the sun from the heat and UV, or may be cast aluminium, steel, or other suitable metal.

The LED lens 404 may for example be a wide beam lens to spread the light from the LEDs 403, or may be narrow beam lenses to focus the light towards the imaging axis.

The 0° polarising filter 406 may for example be a linear absorptive polariser.

The 90° polarising filter 408 may for example be a linear absorptive polariser identical to and mounted perpendicularly to polarising filter 406.

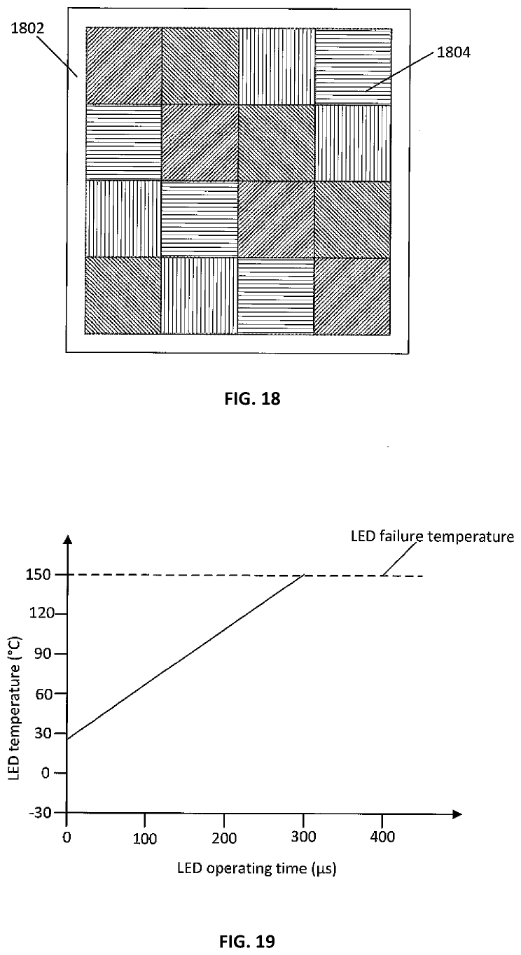

The system can also include several second polarisers for polarising the light reflected from the plant. These second polarisers have different polarisation axes to provide several different views of the plant. Simple cross light polarisation with only one polariser for the reflected light can sometimes miss detail that is visible at certain angles, fungal disease fruiting bodies for example. The different polariser angles can allow for images to be taken with full, partial and no (or minimal) specular reflections to improve detection capability. The second polarisers can be used with a single image sensor, or each polariser can provide filtering to its own image sensor. FIG. 18 shows one such arrangement, with 16 different polarisers 1804 overlaying an image sensor 1802. The polarisers have polarisation axes of 0°, 45°, 90° and 135°. An exemplary device with several polarisers on a single image sensor is the Sony IMX250MZR/MYR polarisation image sensor.

A second colour CMOS sensor may be used in combination with the first colour CMOS sensor to capture stereo photographs, while also using the active lighting and polarisation system already described. This would capture stereo images with the same high quality and integrity, by accounting for the varying lighting conditions of outdoor environments in tall crop management applications. The second sensor would be the same as the first sensor and use the same parameters. Images on the second sensor would be taken at the same time as the first sensor. This could be achieved through an electronic trigger signal from the first sensor, to the second sensor. Both sensors could be referred to as a single “stereo camera”, or “stereo vision system”. A horizontal displacement between the two sensors is required to create two differing views. The stereo camera could be inside one housing along with an over-driven LED package and cross-light polarization. Or, two CMOS sensors could be in separate housing with individual over-driven LED packages and cross-light polarizers. These individual sensor housings could be set at varying distances apart to create an adjustable stereo vision system and a wireless or wired trigger signal might be sent from the first camera to the second sensor. Or, a simulated stereo vision system might be created by using only one CMOS sensor in a housing along with an over-driven LED package and cross-light polarization. Two images would be taken at different positions as a vehicle is in motion. The distance between the images could be calculated by time and the speed of the vehicle, a global positioning system, or an inertial measurement unit, etc. Depth information can be extracted by comparing the relative differences in coordinates between two images to the displacement of the two images. A three dimensional reconstruction of the images could then be produced. This stereo camera could be used for the purpose of calculating and calibrating size of detections. e.g. bunch size, fruit size, flower size, bud size, shoot size, cane size, trunk size, etc. for measuring, estimating, or forecasting yield, fruit, berry, or plant health, vigour, growth stage, ripeness, weight, or other phenological characteristics. An off-the-shelf stereo vision camera may also be used, if the internal sensor has the same or similar specifications as the colour CMOS sensor already described. An example of an off-the-shelf stereo vision camera is the Stereolabs Zed 2.

The illumination device can be one or more LEDs, in this case white LEDs 403. Fifthly the white LEDs 403 may be strobed. Depending on the LEDs used, a strobe mode is possible where the nominal or average input rating eg: 110 W may be achieved by only powering the LED for a very short period in each duty cycle, but to a much higher level without damaging the LED. This allows the lumen output of a relatively cheap LED array to be significantly higher than the sun (>100,000 lux). This in turn may obviate the need for a sun shield, without a commercially infeasible illumination cost.

The white LED 403 may for example be a chip-on-board LED that is high-power (depending on the application a different cost vs. size vs. overall output may be desirable), with a high colour rendering index (CRI), for example 80 CRI, and cool colour temperature, for example 5700K, to preserve real-life colours in captured images. In some cases, the CRI and colour temperature may be selected based on the colour shift induced by overdriving the LED so as to lead to a substantially neutral light when the LED is overdriven. The increase in drive current during on time should be designed up to a maximum based on the rated maximum current increased proportionally to the reduction in duty cycle.

For example, the current could theoretically be increased by up to 50 times the rated current for an increase of up to 100 times the rated electrical power. If the nominal LED voltage is 72V (relatively large high-power LED e.g. Cree™ CMA3090R), during strobe mode, with a duty cycle of 0.06%, the drive voltage could be increased by 3 times the rated voltage to achieve such an increase in current without damage. In other words, empirical testing will be required for a particular design, as with very small duty cycles, the practical maximum current during on time will be less than a proportional value due to other considerations. These considerations may include prolonging LED longevity, keeping LED colour temperature and CRI consistent, whilst maximising light output. These may include high frequency effects, manufacturing differences between LEDs of the same part number, and the package thermal time constant in comparison to the pulse width—if the heat generated during the pulse is not able to be dissipated through the package quickly enough, the LED will fail. As the thermal time constant of the LED package increases as the device gets larger, smaller devices can be driven closer to the theoretical maximum, but a higher number of lower power (smaller) devices may take up more real estate and require a bigger housing, or cost more in total per lumen than larger devices.

So, if the strobe frequency was 8 Hz (period of 125 ms) then a pulse width of 75 us would be possible. This would equate to a practical frame rate of 8 frames per second (FPS) and exposure time of 75 us on the CMOS sensor to illuminate the subject area. In practice the exposure time could be specified first in the design process, according to the maximum design speed of the vehicle carrying the system to avoid blurring.

The LED bond wires and associated power supply must also be able to supply and support the massively increased current, albeit for a 0.06% duty cycle. This may require a low inductance circuit and may also require magnetic shielding to reduce EMI to a regulated level.

Overdriven LEDs can warm up more quickly than LEDs driven at or below a rated voltage. If the heat is not adequately dissipated, the junction temperature of the diodes can eventually get too hot and the bond wires will melt, resulting in the failure of the LED. One way to mitigate this is to only allow the LED to emit light for short durations.

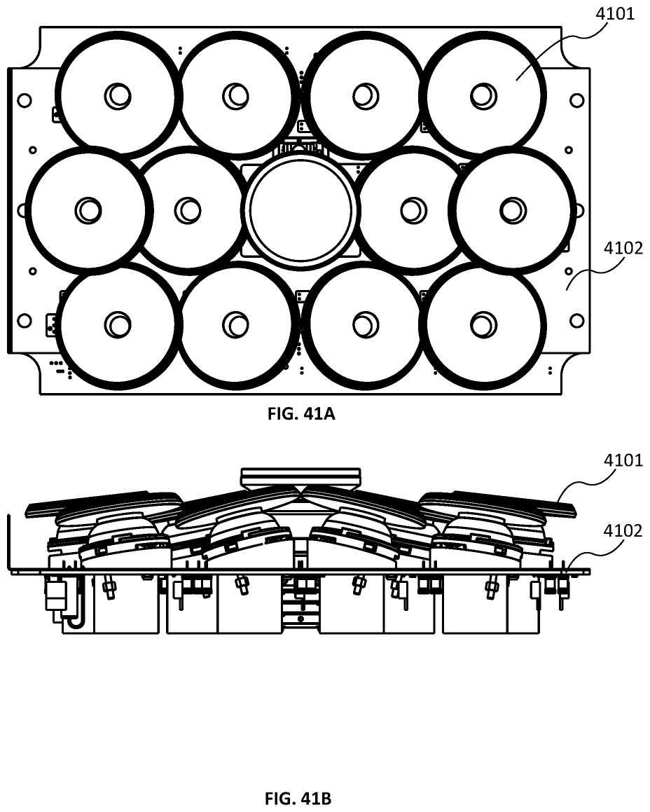

A passive cooling method may be used, such as the example device shown in FIGS. 41A and 41B, which comprises an aluminium heat sink 4101 on each LED, to disperse heat away from the LED package. The interface between the heat sink and the LED may be a thermal paste, glue, or pad to maximise heat conduction. The thermal conductivity of the thermal paste, glue, or pad may be, for example, 1.5 W/m-K. The heat sinks may be used alone or may be connected to another thermally conductive material, such as a copper sheet 4102 with a thermally conductive interface as described above to further conduct heat away from each LED package. The copper sheet may be affixed to the device enclosure, if the enclosure is made of a metal, like steel or aluminium, with a thermally conductive interface between the enclosure and copper sheet. This may allow the heat from the LED package to be dissipated outside of the box.

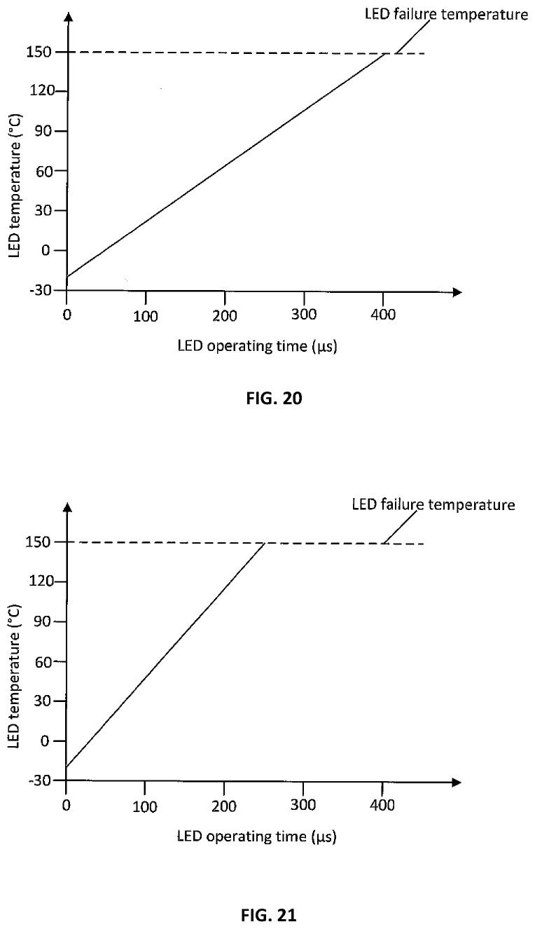

An active cooling method such as a refrigeration system or Peltier cooler can be used to cool the LED significantly below ambient temperature to create a ‘temperature buffer’. This is shown in the FIGS. 19 and 20 discussed below whereby lowering the starting temperature allows the LED to emit light for longer before failure. More time in which the LED is emitting light results in higher overall light output.

Alternatively, if the LED is being flashed for a set duration—200 μs for example—the supercooled LED will have a lower peak temperature than its ambient counterpart. This puts less stress on the circuit over time and may increase the LED's longevity.

Examples of LED operational characteristics are shown in FIGS. 19 to 21. In these examples, a Cree® XLamp® LED is considered, where the maximum junction temperature is typically 150° C., at which point the LED can permanently fail.

In FIG. 19, a non-cooled LED has a voltage 1.5 times greater than its rated voltage applied to it, at a room temperature of 25° C. The temperature of the LED rises until it reaches the failure junction temperature of 150° C. after 300 μs.

In FIG. 20, an LED is initially cooled to −20° C., then a voltage 1.5 times greater than its rated voltage is applied. Assuming a similar gradient of temperature increase to the non-cooled LED, the temperature will rise for over 400 μs without reaching the failure temperature of 150° C. This shows that the cooled LED can be overdriven by the same factor as a non-cooled one for a longer time than the non-cooled one without failing.

In FIG. 21, an LED is initially cooled to −20° C. then a voltage 3 times greater than its rated voltage is applied. The temperature rises more steeply than in the previous examples (FIGS. 19 and 20) and reaches the failure temperature of 1505° C. in 250 μs. Despite the time to failure being reduced slightly compared to the other examples, the light output (both instantaneous and integrated over the operation cycle) will be increased significantly when the applied voltage is increased by a factor of 3. This shows that greater light output can be achieved by cooling the LED.

Additionally, an operational ‘sweet spot’ can be found by balancing increased light output (due to higher overdriving ratio) with increased length of operating cycles and longevity (due to lower peak voltages). For a cooled LED, the light output and/or operating cycle length and longevity can be greater at this ‘sweet spot’ than they would be for a non-cooled LED.

Gain determines the sensitivity of the sensor; however, the higher the gain, the greater the amount of noise in the image, and this may introduce artefacts in the image that can affect detection of issues. To keep noise to a minimum, lower values are preferred. A gain of 0 to 10 may be sufficient.

While white LEDs and a colour CMOS sensor have been described above, in different applications, other wavelengths or bands may be used, such as invisible bands, infrared, ultra violet, other EM technologies such as radar or lidar, or mechanical waves such as ultra-sonic or sonar.

The image capture devices 104, 106, 108 may be configured according to the requirements of the application. The image capture device should be generally parallel to the plant canopy and may be offset by a working distance of 0.4 m to 0.8 m. As spacing between distinct rows of plants may change, a level of adjustability is required for moving the image capture system normal to the row plane. The image capture device should generally be directed at a region of interest on the plant such as the fruit zone, the plant base, the plant woody structure, the leafy canopy, or any other combination. Therefore, a level of adjustability is required in the plane of the row. The image capture device may be attached to different vehicles such as tractors, or spraying vehicles, harvesting vehicles, or all-terrain vehicles. The number of image capturing devices per vehicle may vary from 1 up to 6 or more. This may be to capture more area of specific canopies or to capture numerous canopies.

In FIG. 1 the lower image capture devices 108 may be directed at the fruit 114, the mid image capture devices 106 may be directed at the mid canopy 116, and the upper image capture devices 104 may be directed at the upper canopy 118.

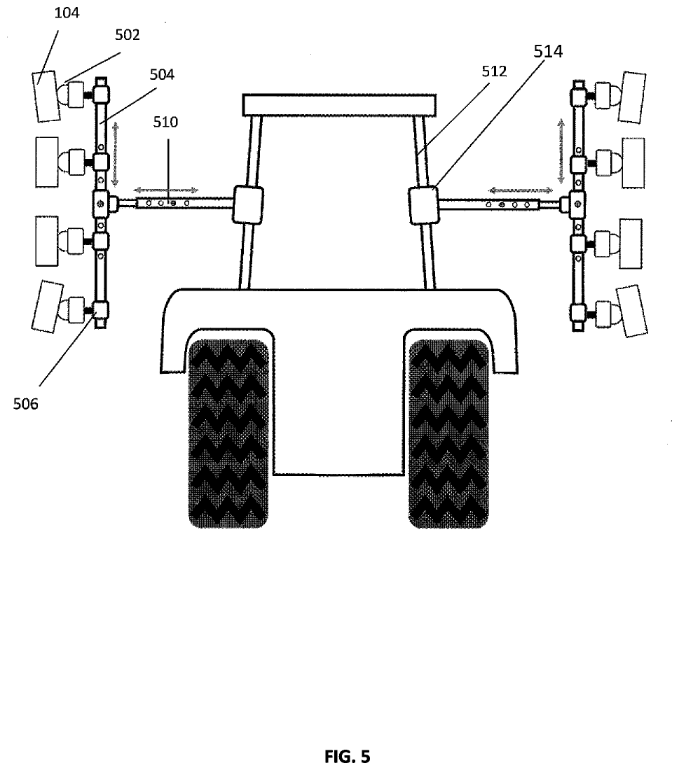

Each image capture device 104 may be mounted as shown in FIG. 5 to achieve the desired camera locations and orientations. The housing 306 includes a ball and socket attachment 502 to the back face which allows orientation adjustment and connects to a generally downward facing shaft 504. Shaft 504 vertically slides within a clamp 506 allowing vertical adjustment. Shaft 504 is attached to a generally horizontal telescoping shaft 510 which is mounted to the tractor frame 512 allowing horizontal adjustment.

Also shown in FIG. 5 are mounts 514 for mounting the imaging system to the vehicle. The mounts 514 can be suction cups. These are cheap and easy to install on numerous vehicles such as tractors, harvester, spray vehicles, or ATVs. They also make the system highly adaptable because they can be attached to any smooth, flat surfaces like windows, plastic walls or smooth painted metal. This may also help install imaging devices in the optimal position or orientation by translating or rotating the suction cup mount. Furthermore, the suction cups can be placed at different locations around the vehicle, such as the front, middle, sides, or rear. Additionally, the suction cup will safely release from the vehicle if the mounted system hits an obstacle without damaging the mounting arrangement or imaging system—a backup catch may be in the form of a cable or tie, where one end secured to the device and the other to some point on the tractor such as around the door handle or wing mirror. In the event that the suction cup fails, the device will be caught by the backup tie rather than impacting the ground and potentially being run over by the vehicle. Suction cups also provide some vibration damping between the vehicle and the imaging system. Suction cups can also be used to mount other equipment, such as a blower, to the vehicle.

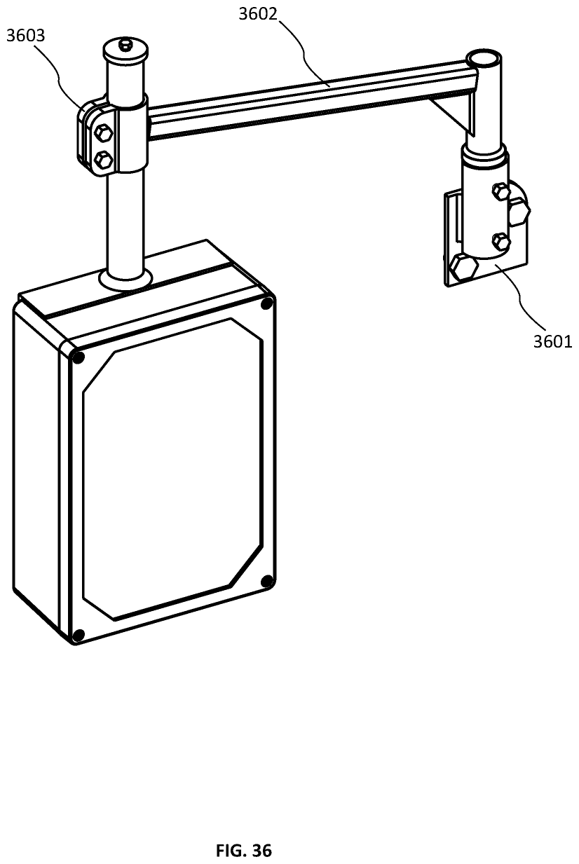

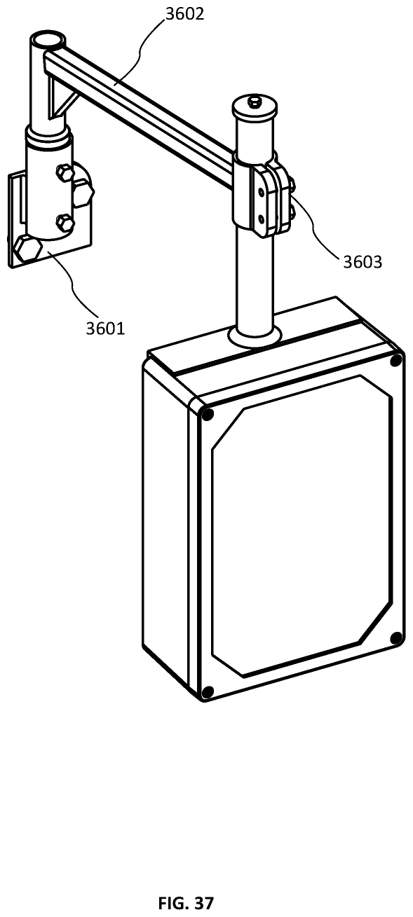

Alternatively, the capture systems may be mounted using a fixed frame that is bolted or clamped to the vehicle rather than using a suction cup arrangement. FIGS. 36 and 37 show an image capture device swivel mount that attaches to a tractor's existing side mounting holes. An example such tractor is the Fendt 211P Vario tractor. The mount consists of a mounting plate 3601 that may change for different vehicles existing mounting holes, a swivel arm 3602 to adjust the distance of the image capture system to the canopy, and a ‘hand’ 3603 that attaches to the image capture system. The hand can rotate to keep the image capture system generally parallel to the canopy and can translate for vertical adjustability. The position of the system could then be secured using bolts, screw knob, clamps, or a locking mechanism. The swivel mount may be attached to existing mounting holes in the middle of the tractor but also at the front, middle or rear of other vehicles or tractors.

In a further embodiment, a fixed frame of box section metal, or round tube/pipe metal, or metal plate or bar may be used to mount an image capture system or systems to an ATV or other vehicle, such as that shown in FIG. 40. The frame 4001 may bolt or clamp onto new or existing mounting holes or existing framework on an ATV or other vehicle. The frame may also be welded to the existing framework or a metal portion on the ATV or other vehicle. The frame may be attached at the front, middle or rear of the vehicle. The fixed frame may set the image capture system to a fixed position with no adjustability that is generally parallel to the canopy. The fixed frame may be adjustable by having different attachment holes to set the image capture system's distance to a canopy or adjust the vertical height of the image capture system. Alternatively the fixed frame may telescope to adjust the position of the image capture system.

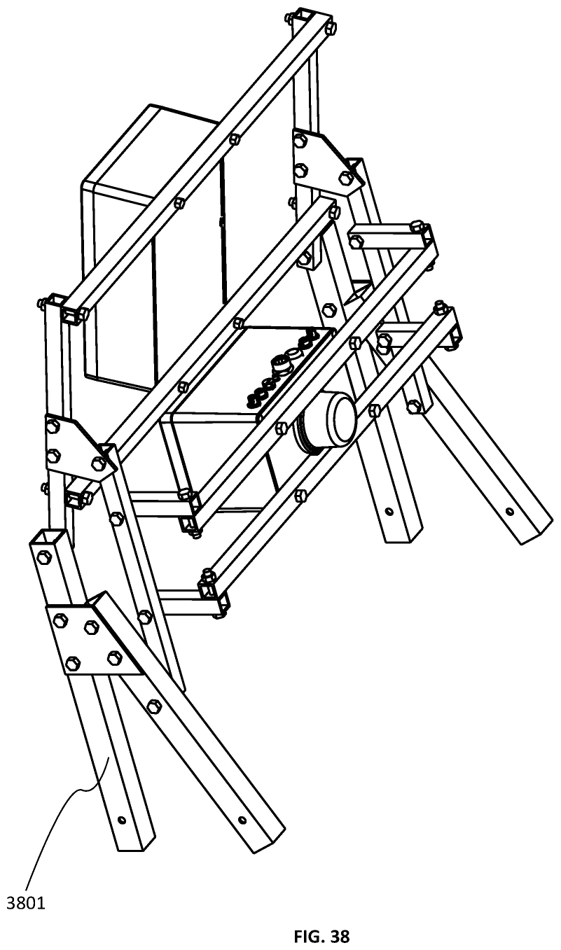

In a further embodiment, a fixed frame 3801 of box section metal, or round tube/pipe metal, or metal plate or bar may be used to mount an image capture system or systems to a harvesting vehicle shown in FIG. 38. In addition to the general use of having the image capture system generally parallel to the vertical canopy for analysing plants, an alternative use case is to have the image capture system pointing in a generally down direction from the top of a canopy. This specific use case when attached to a harvesting vehicle may be used to detect row structural integrity, rather than determining plant health or features, from vibrations as the canopy passes through the harvesting vehicle, since many harvesting vehicles vibrate the plant in order to shake the produce off so it may be harvested. The harvesting vehicle fixed frame may be mounted at the front of the harvesting vehicle or the rear and may be attached via bolting, clamping or welding.

### Blower

At different stages of the growth cycle the fruit or produce may be exposed or it may be covered by foliage. Equally, disease may occur on the undersides of the foliage. For example, powdery mildew and downy mildew exhibit similar symptoms in the early stages; however, powdery mildew exhibits different symptoms on top of the leaf while downy mildew exhibits different symptoms on the underside of the leaf. Mealybug, a devastating pest, lives under the leaves as well. Depending on the application it may be desirable to displace the foliage to capture images of the fruit or produce and/or the underside of the foliage.

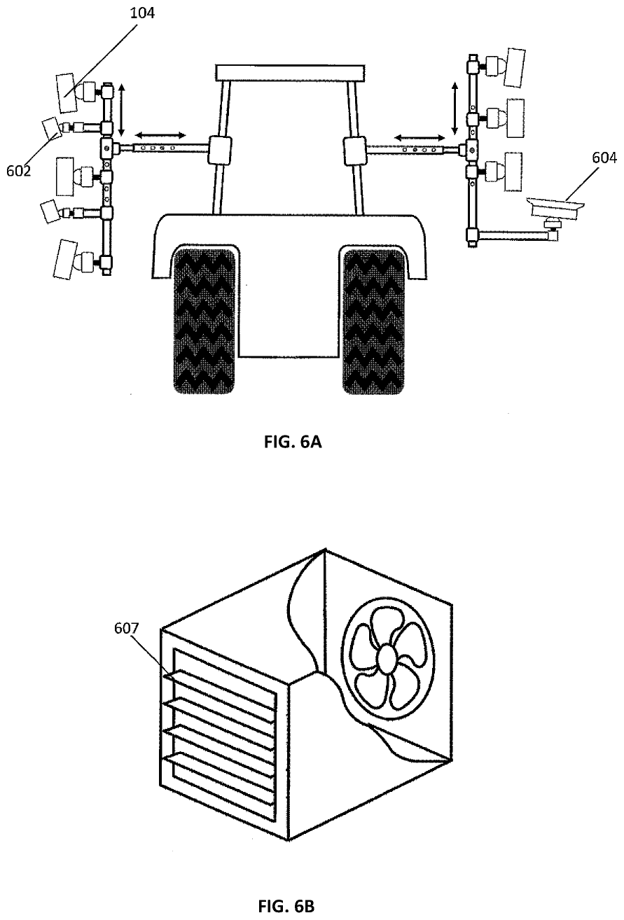

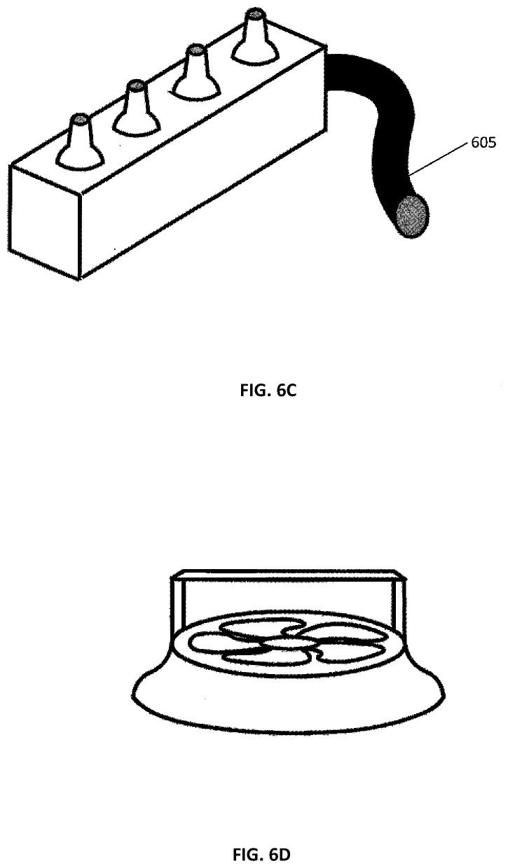

The blower 110 is shown in more detail in FIGS. 6A to 6D. An air pulse arising from below the leaves combined with a co-ordinated camera trigger is used to capture the underside of leaves. Air is likely to be pulsed in short periods as to avoid missing the overside of the leaf during normal collection. To achieve this the blower 110 will be mounted slightly ahead of the camera. The blower will be pulsed so the camera can capture images of the tops and bottom of the leaves (not necessarily of the exact same leaves, but close). In FIG. 2 Blower 110 is shown mounted to shaft 504 through two forwardly extending poles 120 that are perpendicular to, and may slide over, each other. Subsequently the poles are attached to a horizontal shaft and ball socket 122 to the nozzle 124.

The blower may take different forms depending on the requirements of the application. For example, as shown in FIG. 6A, each image capture device 104, may have a corresponding individual nozzle 602 (further examples shown in FIGS. 6B and 6C), or a single nozzle 604 (example in FIG. 6D) may be placed on the bottom to blow the leaves upward, or near the top in reverse to suction the leaves up. FIG. 6C also shows the hose 605 connecting the blower to a source of pressurised gas such as an air compressor.

Increasing turbulence can help to agitate the leaves or other plant parts so that more of their surface, including the underside, can be imaged.

One option is to take a photo of the canopy undisturbed, then generate a pulse of air and time it so that when the pulse disturbs the region of interest the second photo is taken, repeat for each scene. Timing the second photo will often require trial and error so an AI based solution may suit.

A second option is to use two cameras per scene, with some displacement (^{˜}30 cm+). One camera captures undisturbed photos. The second camera has the blower attached to it and constantly captures disturbed canopy photos. Use cm-level geo-tagging to merge the results in post.

A third option is to use a single camera with a single powerful blower with a turbulence grid. Capture and analyse multiple photos per scene, Use cm-level geo-tagging and AI or IMU tracking to merge the results in post. This assumes that most things are exposed by the blower for each scene.

The blower of FIG. 6B has vanes 607 for directing airflow from the blower. The vanes 607 can be tilted in different directions to move the airflow around and effectively increase turbulence of the airflow on the leaves. The blower of FIG. 6E has a grid of slats 606 running in different directions to increase turbulence of the airflow. Alternatively or additionally, the air supply to the blower can be pulsed to increase turbulence.

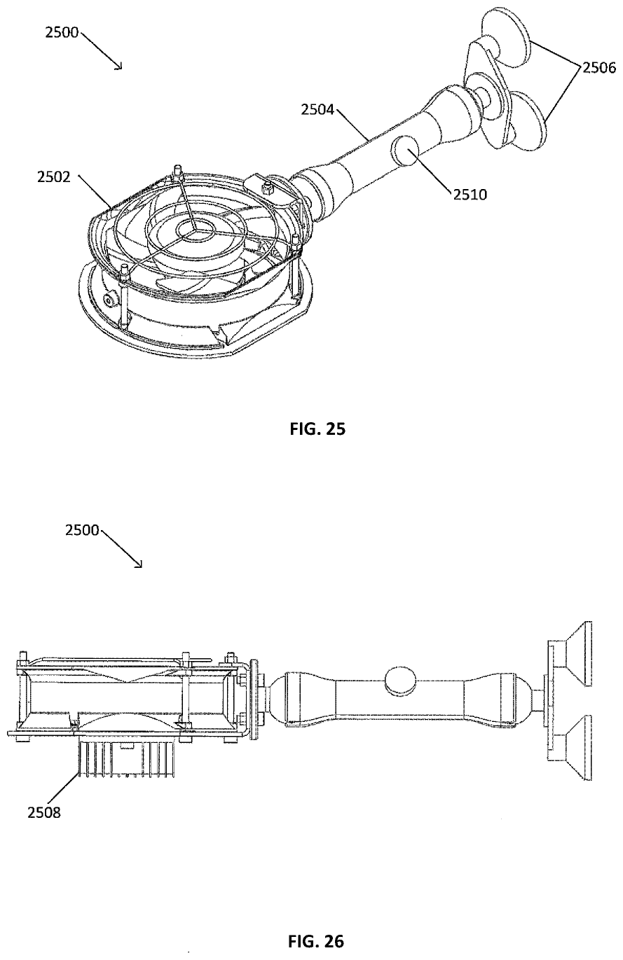

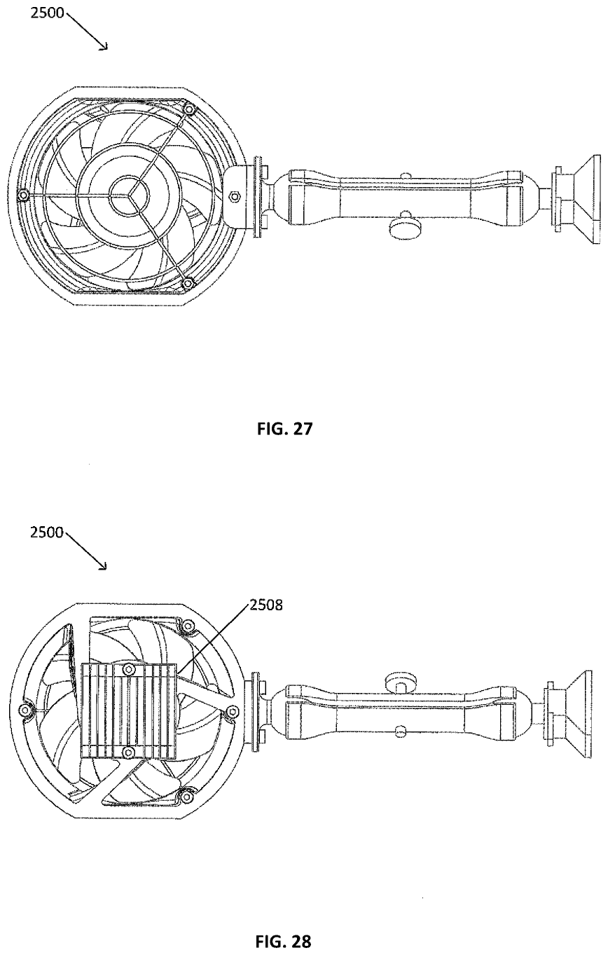

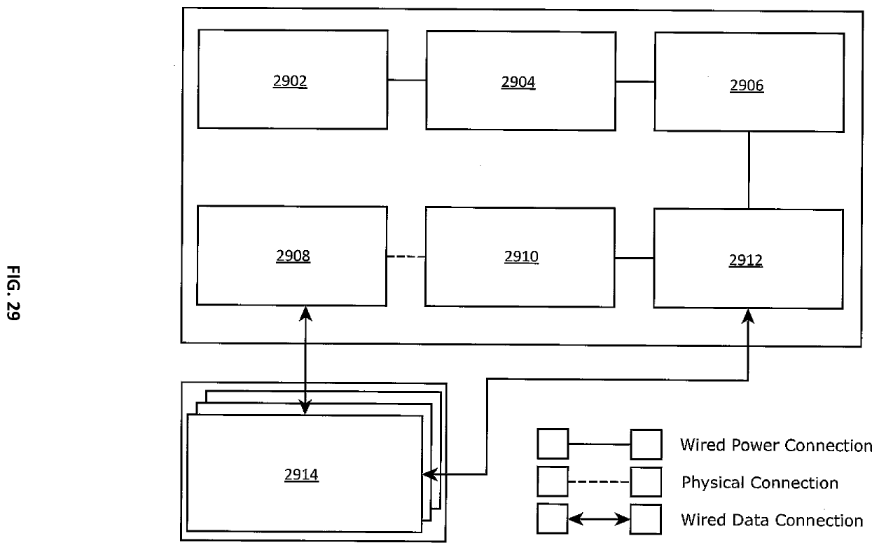

One exemplary blower 2500 is shown in FIGS. 25-28. The blower 2500 includes a RAM® mount assembly with a fan head 2502, mounting arm 2504, suction cups 2506 and a ball joint for attachment to the vehicle and a heat sink assembly 2508. An automotive regulator and boost converter are mounted with a heat sink assembly 2508 and dissipate heat into the blower's air flow using cooling fins of the heat sink. An adjustment knob 2510 is also provided to tighten or loosen the mounting arm 2504 from the ball joints on either end. The mounting arm on a RAM® mount is in two halves (length-wise) and the knob in the middle has a screw or bolt which can then bring the two halves together and tighten the grip on the ball.

Exemplary blower hardware is shown in FIG. 29. This includes a 12V vehicle battery 2902, automotive regulator 2904, boost converter 2906 for outputting 48V, tachometer 2908, fan 2910, pulse-width modulation (PWM) controller 2914 and single-board computer(s) 2914.

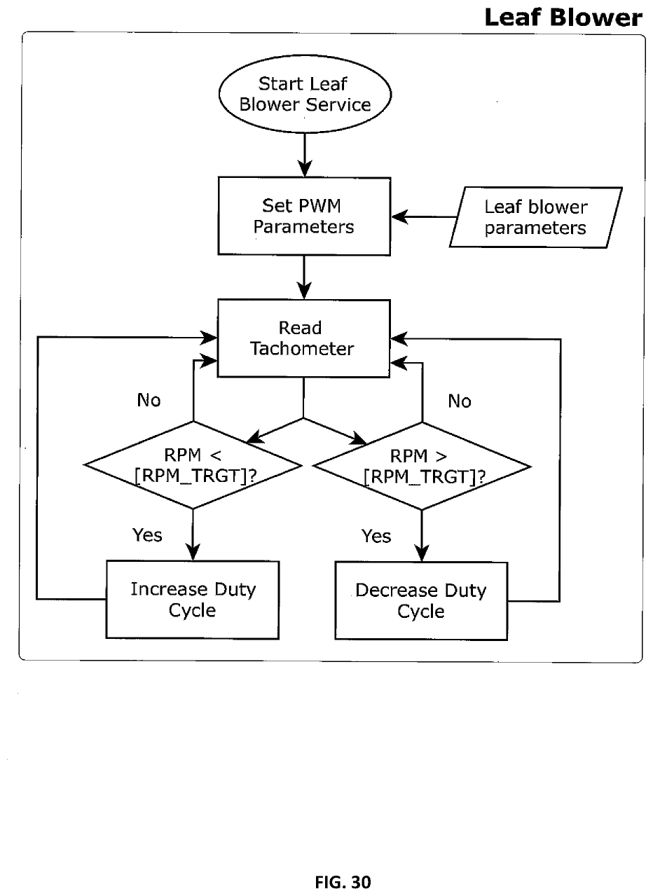

FIG. 30 is a flow chart depicting an exemplary leaf blower speed control algorithm used to program the single-board computer 2914. It uses a closed feedback loop to operate the fan at a fixed Target RPM level. Alternatively it may be pulsed according to a predetermined strategy.

### Sprayer

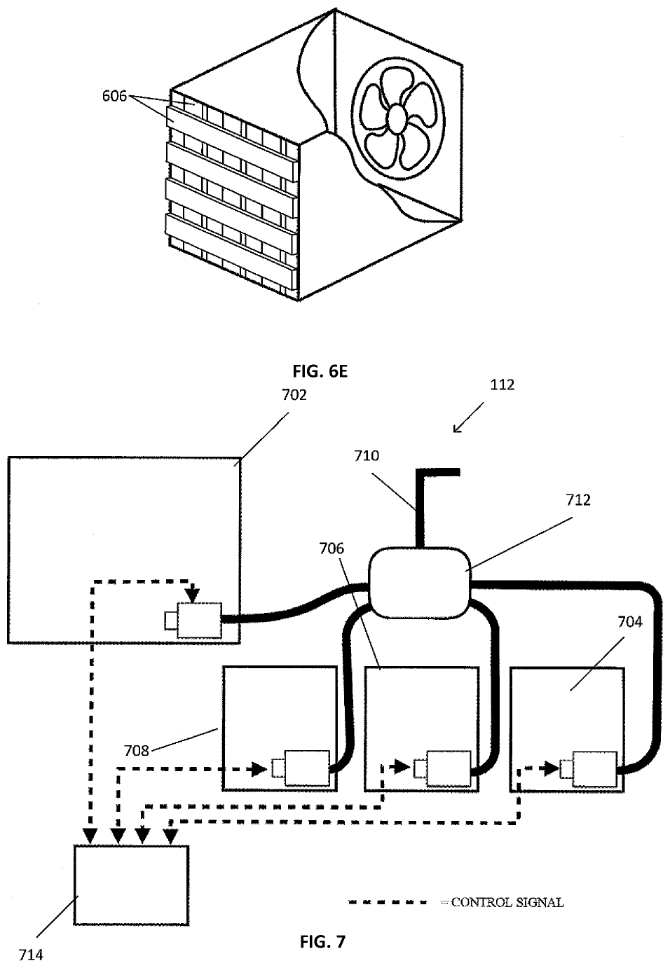

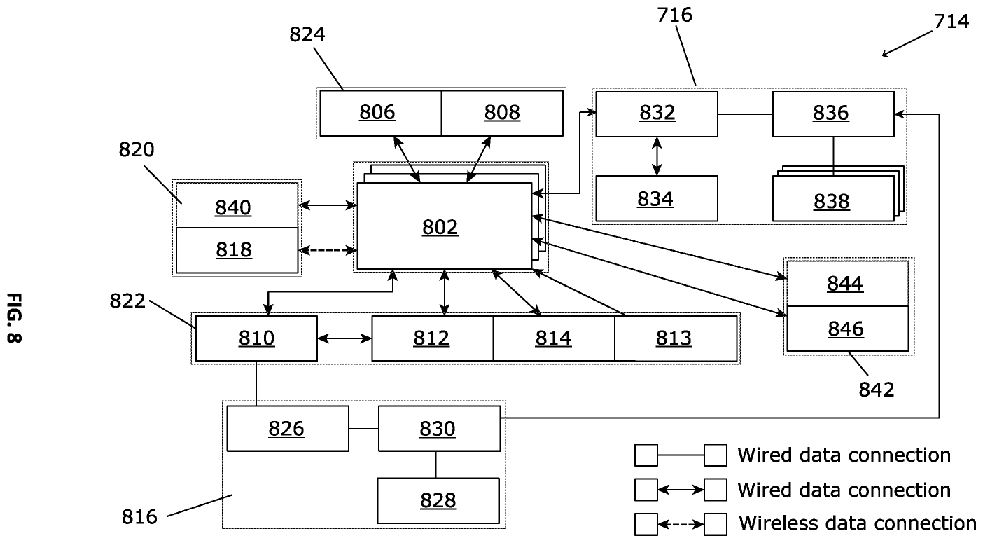

The sprayer 112 is shown in more detail in FIG. 7. Typically spraying in vineyards includes regular spraying of protectant, with occasional spraying of eradicant depending on prevalence of disease or pests. In this case a main tank 702 of protectant, which may be sprayed on the entire crop, can be combined with targeted spraying of specific eradicant, depending on the detection of respective types of disease or pests.

Small reservoirs 704, 706, 708 of concentrated “eradicant” (for each respective disease or pest) may be fed into the main sprayer line 710 (which is usually a common feature across most commercial sprayers), or via a mixing chamber 712. This results in eradicant being sprayed across the canopy over its entire height. Depending on the application requirement, a “diverter” may be used, so that eradicant is sprayed from certain nozzles (via their respective feed lines) of the commercial sprayer based on which camera registers a detection event. In other words, the application of spray to a detection location could either be 2D with the addition of camera height information. Alternatively, the detection location may be specific to a particular part of the plant, foliage or fruit, and directed application of spray or choice of spray may depend on that detection location and/or the type of disease or pest detected.

There may be a detection controller 714 that determines the location of disease and/or pests. The detection controller 714 may emit a control signal(s) to a sprayer controller 716 associated with the sprayer 112 or an appropriate interface. Alternatively, if the sprayer 112 is integrated with the illumination source, multiple image capture devices 104, 106, 108 and/or blower, then a single controller may be employed. The control signal(s) may do the following:
Turning certain nozzles on/off and/or adjusting speed of the fans.
Actuating independent motorised pumps in the smaller reservoir(s) to feed an appropriate amount of treatment chemical(s) into the main reservoir line or mixing chamber.
Actuating independent motorised pumps in the smaller reservoir(s) to feed an appropriate amount of treatment chemical(s) into individual nozzle/fan lines for extremely targeted spraying.
Synchronising the spraying/pumping actions with the detection system.

The exact implementation may depend on individual growers' current arrangements; some growers may already have a main-secondary reservoir arrangement, thus the auto sprayer may interact with the existing spray controller. Other growers may not have such an arrangement, and retrofitting one or more secondary reservoirs to their existing sprayers may be required.

The controller 714 may orchestrate the different actuations in order to synchronise the illumination, air pulse, capture, and spraying. Additionally, location data may be stored as the system 100 travels through the field, including GPS, acceleration, and/or orientation. This data may be used to program a buffer zone of any size (within the horizontal distance from the camera to the sprayer nozzles) for the sprayer, for example, the sprayer may be actuated to spray for 1.5 metres horizontally on either side of the infection site.

Avoiding a covering to block the sun means that crops that cannot be covered in a practical manner can be scanned. To achieve the same effect (over-powering or removing the effects of the sun in the image), the thin canopy crop would need to be sufficiently covered on all sides to block out the sun; examples of such crops may include grape vines or apples grown using a 2D growing system. For a vertical crop, this would require a large tent-like structure, with flexible openings for the canopy to pass through, or may be impossible entirely if the thin canopy crop is grown with the canopy in a horizontal or angled fashion where it is composed from many different plants to form a large thin and uniform canopy.

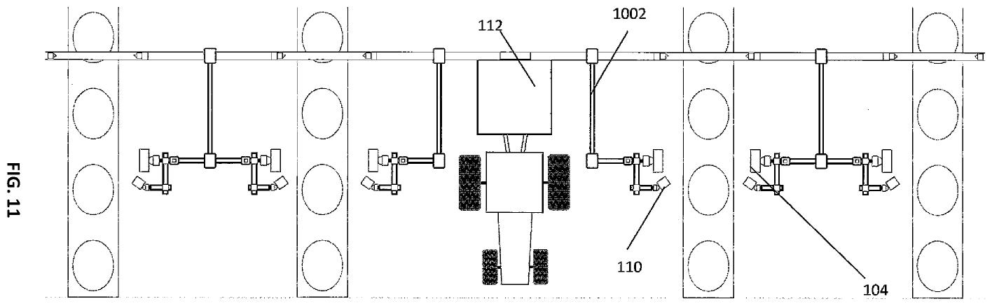

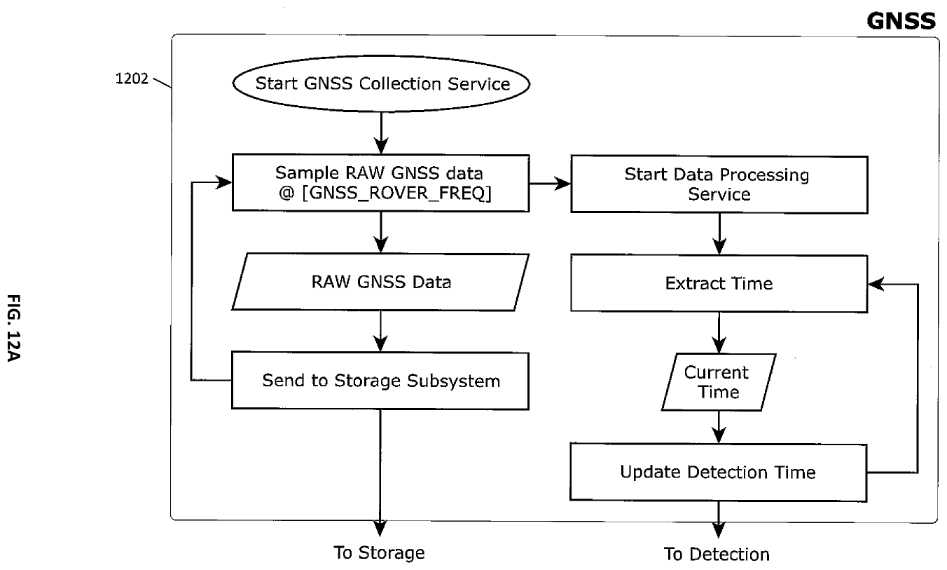

In the case where employing a cover is possible, a camera and lights may be placed under the cover to capture images. The issues likely to be encountered with this approach are:
The flexible openings on the tent may break off or damage soft fruits.
Existing sprayer infrastructure for tall vertical crops usually reaches over the top of a row and/or reaches the other side of the row to ensure good spray penetration, and occasionally spans multiple rows. Sprayers are also generally mounted to the back of a tractor. A large cover would interfere with this spraying process because it couldn't be mounted on the back with the sprayer, and it would be too large to mount on the front without impeding the view of the operator. In the case of putting a cover over the sprayer itself with cameras and lights beneath it, it would become a fumigation tent and the spray would impede the view of the camera. An example implementation for a multi row sprayer is shown in FIGS. 10 and 11. In this case both the image capture device 104 and the blowers 110 are mounted to poles 1002 on the sprayer 112 which extend from the sprayer 112.
Most crop management machinery is mounted to the back of the tractor/vehicle, and again a large cover would impede these processes. A large attachment would not be able to be mounted on the back with other large machinery.
It would be time consuming to install/remove the cover on a day-to-day basis (e.g. switching between multiple tractors/vehicles depending on what crop management operations are to be performed). It may be more appealing to consumers if the device is smaller and easy to install/remove.

In order for spraying to occur simultaneously with detection, the detection process must be done in real time. Real time in that case depends on the requirements of the application. In practical terms, detection and subsequent drive signals for the sprayer must be completed prior to the time it takes for the sensor to image the pest/disease location, until the sprayer nozzles reach the same (or a close) location. This may in some cases require a maximum speed limit of the vehicle.

Alternatively, spraying may occur separately from detection. For example, in a vineyard, protectant spraying is done every 7-10 days and is largely time based, although some growers spray more often following bad weather. In that case, following the vineyard example, the detection data may provide one or more advantages.

### Optimising Protectant Spraying Based on Pest/Disease Pressure

Organic growers cannot spray most eradicants as they're largely synthetic products, and organic based eradicants are more expensive and destructive than synthetic ones. Consequently, organic growers opt to “double-down” on protectant spraying when a pest/disease is detected. This translates to both higher concentrations, and a lower spraying interval.

In large vineyards, for example, growers sometimes tighten their intervals instead of spraying eradicant as the agrochemical cost is very expensive per hectare for eradicants.

In general, information regarding pest/disease presence can optimise the process of protectant spraying so that it's not based only on time intervals.

For example, a large vineyard may choose to halt protectant spraying for another few days upon learning that the disease pressure is very low, and that weather is good for the next few days. Or that they may have to spray immediately upon learning that disease pressure is high and that coming weather is unfavourable.

Similarly, an organic grower may decide to “double-down” on more actively infected areas of the crop.

### Aid Crop Valuation

Wineries, for example, check their contract grower's vineyards manually near the end of the season; if the grower has 3-5% or more of a powdery mildew/botrytis/downy mildew infection, then the crop is devalued or even rejected entirely.

The process of estimating how much of the crop is affected is done manually by human scouts and suffers from small sample sizes (scouts can't inspect every single vine) and human error in the detection of pests and diseases.

Using one or more embodiments of automated detection may provide historic pest/disease progression information right from the start of the growing season until harvest, thus eliminating any human error and resulting in the objective evaluation of a crop's worth.

### Spray Plan Efficacy

Given the lack of coverage by scouts, growers cannot tell if a particular spray plan is successful or not. A spray plan to treat an active infection takes about two days to formulate, meaning the size of the problem area is overestimated to account for diseases spreading during this time.

Using one or more embodiments of automated detection may allow growers to see infection trends over time down to a single vine. This information helps to create efficient and precise spray plans that reduce costs and increase sustainability by reducing agrochemical use.

### Log Pest and Disease Occurrence and Treatments

In some places, the only spray evidence growers are required to provide to wineries or regulatory bodies are spray diaries indicating when and what was sprayed. Such honesty-based systems are prone to human error and exploitation. Growers emphasised that as technology evolves, the level of scrutiny and proof for spray evidence and justification will also significantly increase. Most growers are wary of European Union's actions in “cracking down” on agrochemical use, which is forcing (through market access restrictions) other countries to stand in line.

Using one or more embodiments of automated detection may allow thorough and accurate justification for spraying, and geo-tagged dispensing of eradicant. This may allow growers to provide evidence to winegrowing regulatory bodies, down to individual vines. Growers can spray to optimise the health of their crop without worrying about breaching regulations.

### Crop Data and Visualisation

The detection data collected by the described system may provide the following benefits to growers:

### Creation of a Digital Twin of the Crop

Collecting precisely geo-located information points on a crop would allow each detection to be associated with an individual plant. This includes giving each plant a precise geo-location, and then each plant may have a virtual profile which shows all the information points associated with it, including if it is suffering from pests or diseases, which treatments were applied to it, how many fruits it has, the colour of the fruits or their respective size, identifying damage or blemishes on the fruits, the canopy structure, how dense the canopy is, how many canes, shoots, cordons, or flowers and types of flowers it has, locations of missing, damaged or dead plants so they may be replaced, and any other information related to the plant's health, performance, or outputs. These plant profiles may be accumulated to represent a larger area of the crop such that the grower can easily identify pressing problems which need to be fixed promptly. This may be recognised as a digital twin of the crop. The advantage of a digital twin is that software or machine learning/artificial intelligence processes and techniques can be used to easily analyse the digital twin crop, identify trends, and make recommendations or predictions to growers.

Once enough data points are collected for a plant, and any supplementary data which may affect plant performance, such as weather or humidity data, trends can be established in how each feature/data point changes over time. This would allow the system to make recommendations to growers for best plant management practices. This can extend further into establishing common trends in how each plant changes over time and making predictions of which problems will likely arise before they become significant.

The digital twin may also be used to plan crop management operations by growers, where a visual representation of the digital twin is displayed on a software platform. This software platform may have a user interface where a grower may view specific plant profiles on a map of their crop, specific data layers on a map of their crop, view trends and changes in the crop, download or upload data, download generated work reports to show workers which problems need to be fixed and where, etc.

Data from the digital twin may be used as an input into autonomous crop management machinery, such as self driving tractors that are equipped to act on recommendations made by the system to look after the crop without human involvement.

### Predict and Forecast Crop Performance

By collecting pest and disease information, yield information, and localised weather information over time, machine learning models or statistical analyses can be used to identify trends and correlations between yield, pest and disease pressure, and other environmental factors. These trends and correlations may then be used to predict pest and disease pressure or yield in the future.

Traditional yield estimation techniques involve systematically sampling across the crop for evidence of crop growth. This is done by dividing the crop into regions and sampling each one. In early stages this can include counting buds, shoots, and more. In later stages this can include counting inflorescences, bunches, fruit and more. This data alongside historical ground truth data of average weights and sizes is used to extrapolate an estimate of yield for each sampled region and then combined to give a total estimate for yield. This sampling is done periodically throughout the growth of the crop, often with weeks or months between sampling. The error in traditional yield estimation techniques is caused by a sampling error both temporally and spatially as well as human error during counting.

The described method decreases the sampling error caused in both respects. The vision and detection system allows for precise counting at a per plant level which can be synergised with traditional yield estimation techniques to decrease the error caused by bad sampling. This method can be applied at a higher rate which collects more data to aid with extrapolating yield estimates and removes a large component of the human labour required to count.

Combined with the ability to detect pests and diseases, or other inputs like irrigation or fertilisation, the described method is able to precisely identify areas in the field which have had their growth stunted, damaged, or for significant diseases, areas where fruit must be removed by human intervention. This allows for calibration of the estimated count of a crop at a precise location and time.

A combination of this data and data from other sensors can be used to forecast with higher precision. Current methods either use distant weather stations or little equipment at all to gauge key variables that influence yield and disease risk. Temperature and humidity play an integral role in disease forecasting. The described system allows for measurements of small variations among microclimates that are known to be created in precise areas across the crop. One approach to yield estimation is to count fruit or bunches of fruit, or inflorescences/flowers and young shoots which will eventually grow fruit on them. Counting inflorescences and shoots is a more predictive method of estimating yield, as it is difficult to know how many inflorescences will actually mature into bunches. This method may require some statistical modelling and historical data of harvest tonnage which is correlated to the number of shoots/inflorescences on each plant.

Counting bunches and fruit may be a more concrete way to estimate yield, and the closer this is done to harvest, the more accurate it will be. Things which can affect yield once the fruit have matured are pests and diseases, so some predictions on how these problems may affect the final yield needs to be done in case there is fruit which must be discarded.

Estimations of how many bunches or fruits there are per plant can be correlated to their mass, so the total tonnage can be estimated at harvest. This would need to be done by taking sample measurements of the fruits and correlating their size and shape to their weight. From there, the size and shape of a fruit may be estimated from the images (including estimating how big it could be if it is partly occluded) and then estimating the weight.

This information may help growers to:
plan where to discard or drop produce which is unsuitable for harvest
plan where to drop produce deliberately to control the size or nutritional content of remaining fruits, or to control yield overall
view crop output over time
plan supply chains for transporting harvested produce
estimate the value of the crop before it is harvested
create a health history for every plant, block of plants, and the crop as a whole.
receive warnings about plants that are underperforming or at-risk of developing a health problem before it actually occurs.

### Controller

Because the LED is being strobed, the exposure time configuration, and the physical distances between the moving components, in some applications it may be desirable to have a careful synchronisation between the LED strobe, blower pulse, image detection, and spray application.

The synchronisation and other control functions may be implemented using the detection controller 714, a more detailed example of which is shown in FIG. 8. The controller 714 includes a microcontroller, single board computer (SBC) or processor 802, memory,, an inertial measurement unit (IMU) 806, Global Navigation Satellite System (GNSS) or Global Positioning System (GPS) 808, LED flash circuit 810, CMOS sensors driver 812, blower controller 814, power supply 816 and spray controller 716.

The SBC or processor 802 is in communication with storage 820, which can employ external storage 840 and a user's phone 818. The LED flash circuit 810, CMOS sensors driver 812, blower controller 814, and weather sensors 813 make up a collection subsystem 822. The power supply 816 includes a 12V or 24V vehicle battery 828, a boost converter 826 for outputting 200V, an automotive power regulator 830 for outputting 12V or 24V and optionally a buck converter for outputting 5V. The IMU 806 and GNSS/GPS unit 808 make up a positioning subsystem 824. The boost converter may be isolated from vehicle battery 828 for electrical safety, or to reduce interference between it and low power components such as the SBC and signal lines. An automotive power supply may also be used to prevent voltage spikes from the vehicle battery from damaging components or the SBC. The spray controller 716 is made up of a sprayer controller 832, a host sprayer controller 834, a pump driver 836 and one or more reservoir pumps 838. The SBC is also connected to a communication subsystem 842 that includes a cellular module 844 and a module 846 for Wi-Fi and/or Bluetooth communications.

Weather sensors (eg, to monitor temperature and humidity) can help forecast pests and disease pressure more accurately for each block/section of the crop rather than relying on a regional weather forecast, or even to a finer resolution within each block/section. This means pests or diseases may be identified at an earlier stage, or smaller problem areas may be flagged as high-risk and the grower can take pre-emptive actions like applying a preventative chemical, trimming canopies to reduce humidity, etc.

In the case that multiple CMOS sensors are utilised and one or more of these sensors are mounted on either side of the vehicle, it will be necessary to distinguish which sensors are on the left side, and which are on the right side of the vehicle. This is because when data is plotted on a map, it needs to be overlayed onto the correct row in the crop. One way to achieve this is to have distinct plugs for the left and right cameras into the controller box, and relying on the user to install the boxes in the correct position on the vehicle. Another way to implement this is using computer vision or machine learning to analyse a number of photos taken while the vehicle is moving. When the vehicle reaches the end of a row to turn around, one camera will be able to see an end post or be closer to the crop than the other, meaning the direction of the turn may be identified from the images, and therefore the system may identify which camera is on the left or the right.

The CMOS sensor may output a digital signal to indicate when the exposure is active (so it will be high for the length of the exposure time, e.g. 20 us), and this may be used to trigger the flash circuit (so in this case, the camera is “software triggered”, and then the flash is triggered by the camera). This signal can be used to trigger or synchronise any actuation, it is not necessarily specific to activating a flash. The sensor's internal delay between the exposure being active and the control signal appearing at the output may vary between different sensors, but is generally in the range of microseconds. The CMOS sensor may contain a signal mode whereby an output is activated prior to the exposure being enabled. Such signal can be used for enabling the flash, and the signal on-time and pre-emption timing can be configured appropriately.

The synchronisation of the data capture and flash may also be implemented using a “hardware trigger”; one signal can be generated by the computer to trigger the camera and the flash, or two signals with a programmed delay can be used (e.g. to make sure the flash is on just before the exposure starts).

The flash mechanism requires time to reach the on state; this is called ‘rise-time’. Rise-time comprises the time required to charge the MOSFET or IGBT gates fully, and signal propagation delays through other logic components of the circuit. Optimal image capture is achieved when the sensor is exposed after the flash is fully on. Therefore, there is a delay between when the flash is enabled and when the sensor is exposed. For example, a rise time of 500 nanoseconds may be suitable.

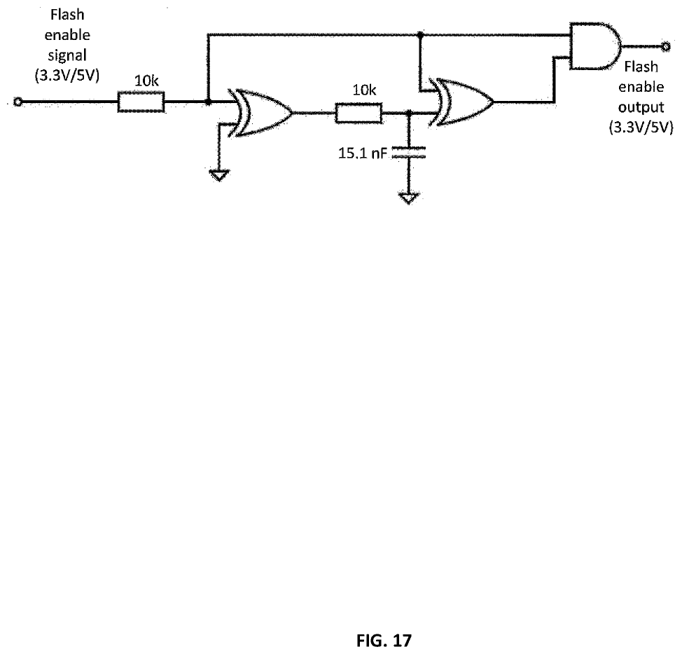

The signal to the LED may need to be “filtered”. An example circuit to filter the signal is shown in FIG. 17. The filter may be used to protect the LEDs from accidental over-triggering, e.g. if the camera's GPIO malfunctions, or if the exposure time is set too high (assuming the CMOS sensor triggers the LED), or if there are hardware or software glitches. If the control signal is high for longer than some specified time, e.g. over 300 us, then the filter will pull the control signal low. The example in FIG. 17 uses AND and XOR logic gates and an RC filter, but the same result could be implemented in other ways depending on the requirements of the application e.g. using a 555 timer, in software, or using other configurations of logic gates/circuits.

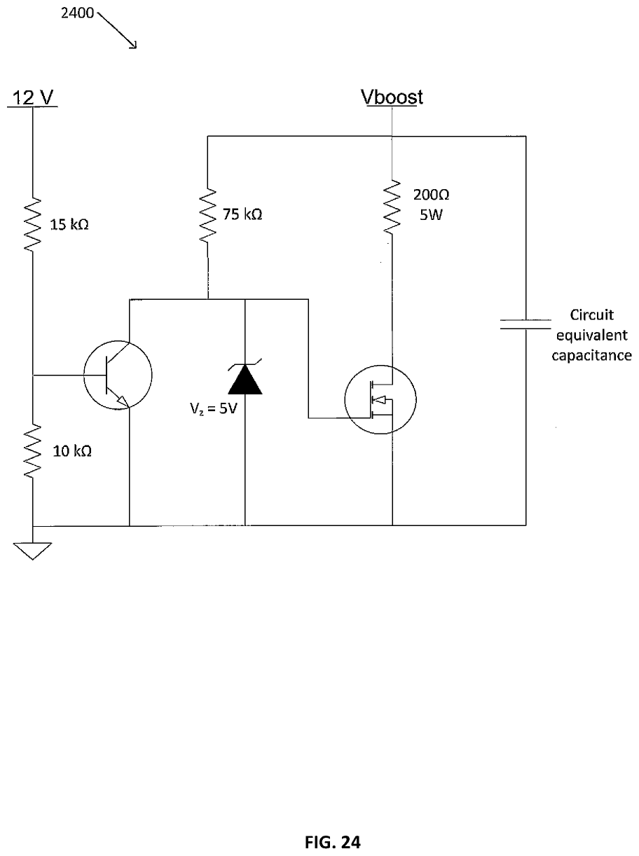

A safety discharge circuit may also be present in the enclosure to make sure that when the main power is disconnected or the system is powered off, any stored energy in the flash circuit is discharged to a safe level. An LED may be included in the circuit to indicate the presence of hazardous voltage or stored energy, and may turn off once the stored energy is discharged to a safe level. An example safety discharge circuit 2400 is shown in FIG. 24. There may be multiple ways to implement a safety discharge circuit, including using a depletion mode MOSFET, mechanical switch, relay, or with a high-power bleeder resistor to dissipate the stored energy.

The air blower's purpose is to blow the leaf and show its underside for a subsequent image following an image capture of the overside of the leaf. There is a delay between when the air exits the nozzle and reaches the leaf; this delay is determined by internal factors such as exit velocity, and external factors such as surrounding wind. Ideally, the delay is kept to a minimum, thus collecting a similar photo of the overside/underside. The delay between the first photo and second photo can be minimal such that the two photos largely overlap each other. The first photo capturing the overside of the leaves, and the second photo capturing the underside following the air pulse.

The air pulse may be delivered pre-emptively such that the first photo is taken before the burst of air reaches the leaves, and that the second photo is taken just after the air reaches the leaves. In this case, the leaf blower may be activated 100-200 ms before the first image capture, so that by the second image capture the leaves are displaced by the air, therefore, showing their underside.

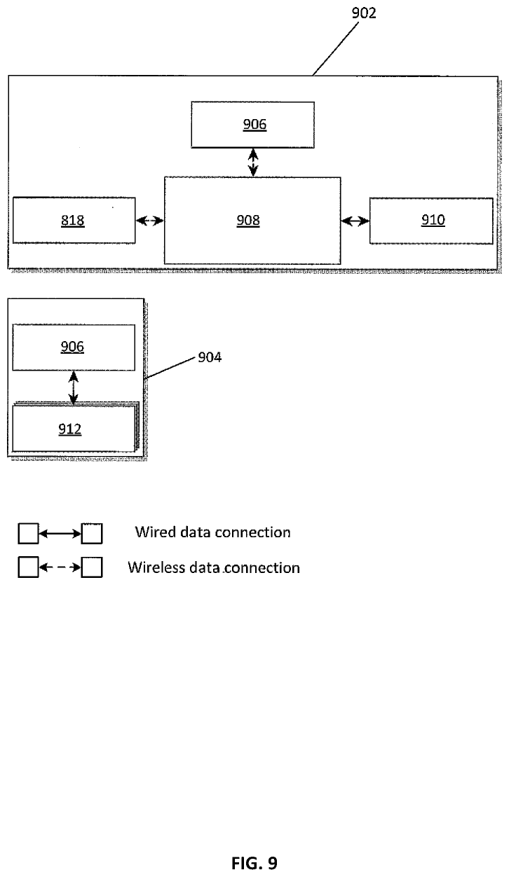

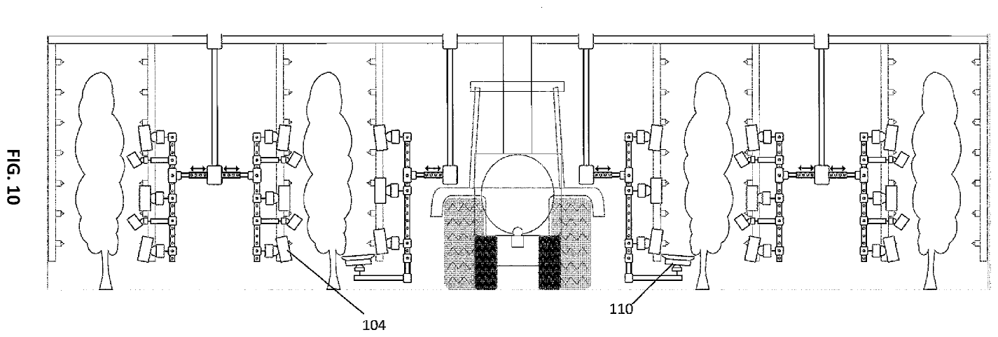

The controller 714 may communicate with a mobile device 818 or base station 902. The mobile device may include an app designed to transfer the processed data from the rover to the base station 902 for uploading to the cloud 904 via the internet 906, or inputting system parameters such as the buffer zone, or relative positions of system components. The base station 902, shown in more detail in FIG. 9, includes a single-board computer (SBC) or microcontroller 908, and GNSS 910 which is primarily to aid with generating more accurate location data from the GNSS 808 of the rover, and store that together with all other data collected into the cloud 904. Cloud storage and retrieval can be provided using server cluster 912.

Low-power wide-area network technologies such as LoRa or Sigfox may be used to establish a communication link between an internet connected base station and a(the) mobile unit(s) for the purposes of transmitting detection and spray events in real-time to the cloud 904 and/or exchanging GNSS correction information for executing real-time kinematics (RTK) to provide centimetre-level positioning capability for the mobile unit.

RTK requires carrier-phase GNSS information from both the base and the rover. This information is gathered at a certain frequency. The base is stationary while the rover moves; therefore, the rover often operates at a higher frequency than the base. For example, the base may operate at 1 Hz and the rover at 4-10 Hz

One option is to use LoRaWAN wireless communication between the base station 902 and the controller 714 to achieve a good real-time kinematic (RTK) location solution. Alternatively, a post-processing kinematic (PPK) solution can be implemented whereby the RAW GNSS measurements can be stored on the tractor/ATV unit (alongside detections) and then uploaded to the cloud over the user's phone (camera system <---Bluetooth---->User phone<----Wi-Fi----->Cloud) or through a USB drive (camera system <---USB--->flash drive <---PC---->Cloud). The base station may be positioned within 10 km of the rover and is connected to the internet via Wi-Fi/Ethernet to constantly send RAW GNSS measurements to the cloud. The kinematic calculations are done in the cloud to get absolute co-ordinates of the rover's movement over time.

Bluetooth and/or WiFi connectivity may be used to transfer data from controller 714 to the cloud 904 either directly or via an intermediate device (smartphone, laptop, base station, Wi-Fi Repeater—an alternative to a radio link and applicable if controller 714 is within WiFi reach). For the purposes of: Transmitting detection and spray events to the cloud and/or exchanging GNSS correction information for executing post-processed kinematics (PPK) to provide centimetre-level positioning capability for the mobile unit and/or for sending evidence images to prove the detection visually to users.

Another option is to transfer data from controller 714 to the cloud 904 through cellular means. This includes but is not limited to 4G, 3G, 2G, and LPWAN technologies, such as CAT-M1 or NarrowBand IoT or others. For the purposes of: Transmitting detection or spray events to the cloud and/or exchanging GNSS correction information for executing post-processed kinematics (PPK) to provide centimetre-level positioning capability for the mobile unit and/or for sending evidence images to prove the detection visually to users.

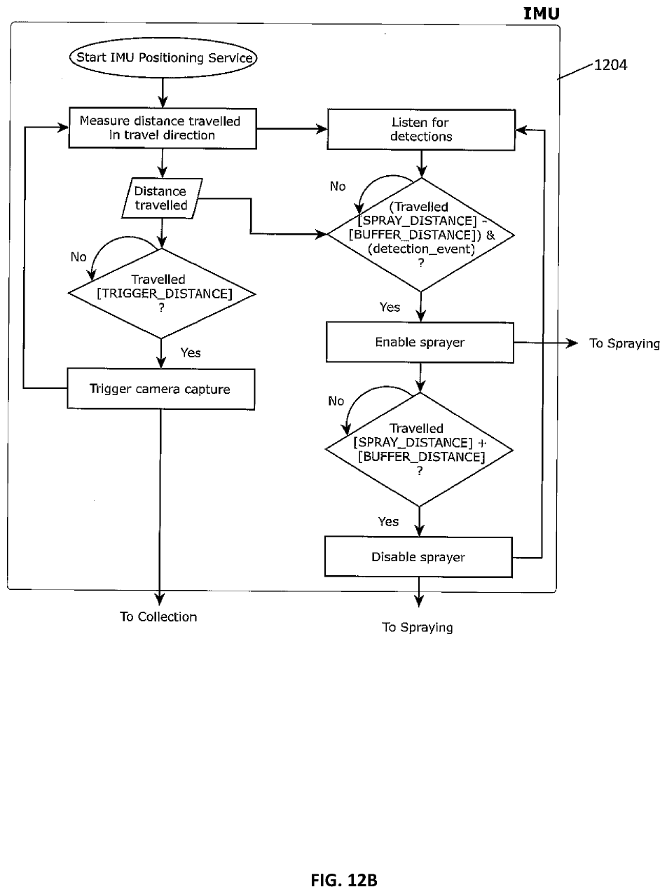

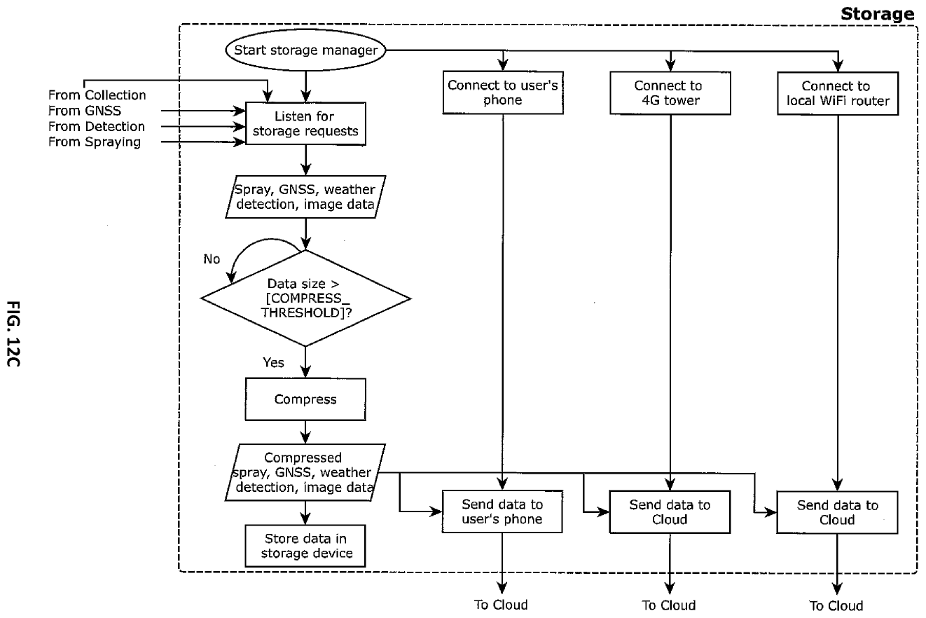

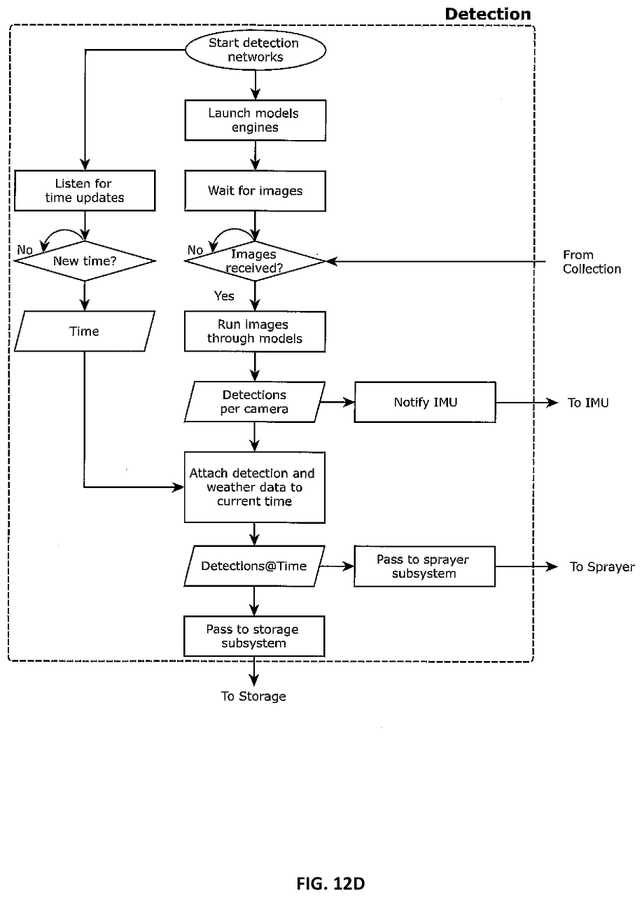

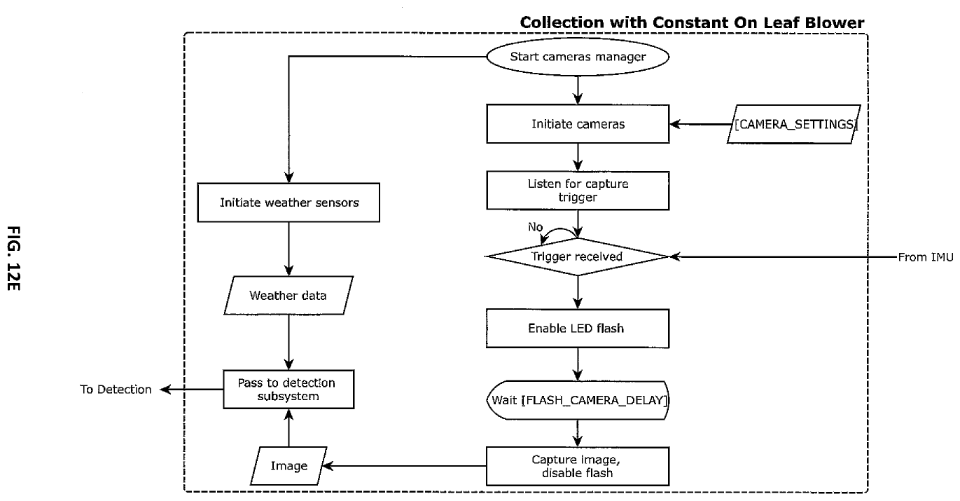

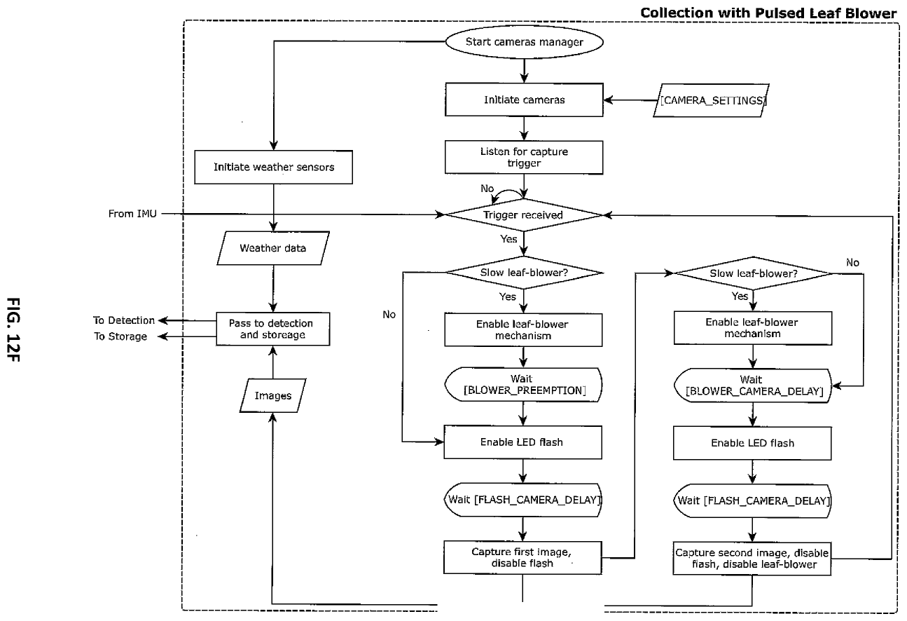

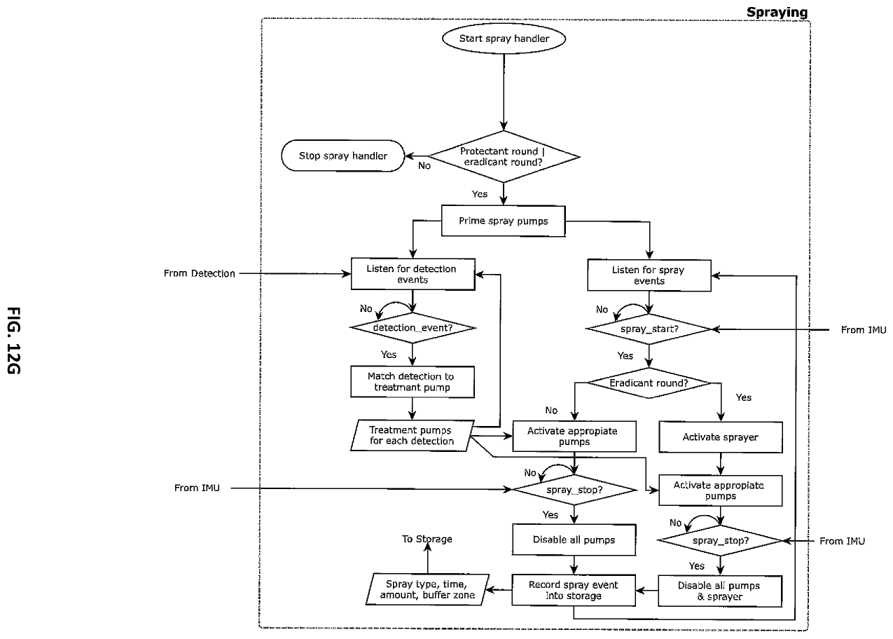

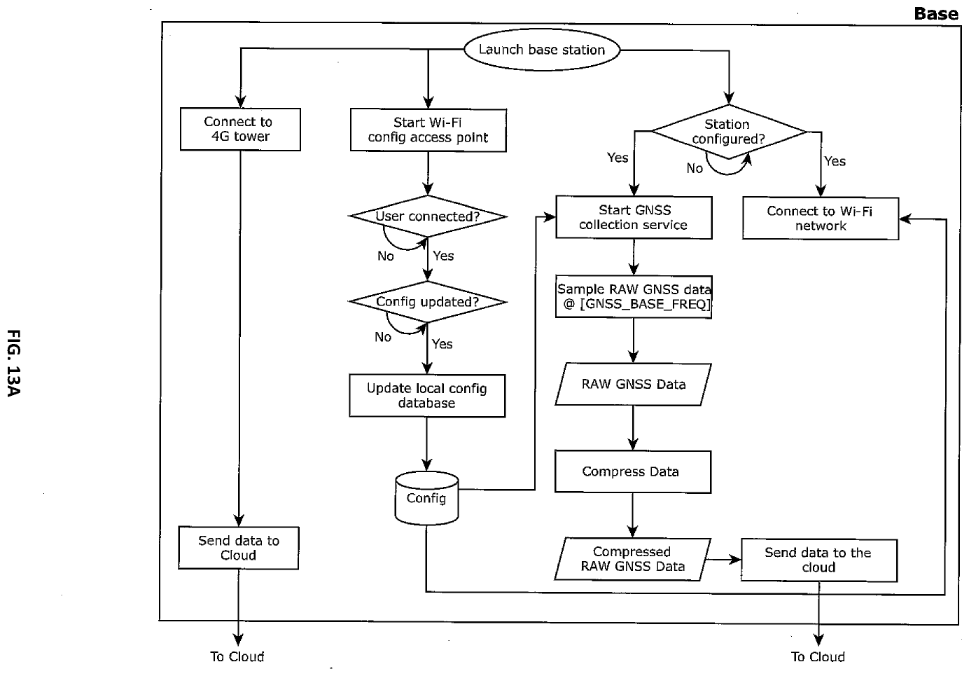

FIGS. 12A-12G show the process followed by the controller 714 to determine location, to collect the image data, to detect pests or disease in the images, to control eradicant spraying, and store the data. FIGS. 13A-13B show the process followed by the base station 902 to acquire and transmit the location data and by the cloud 904 to process the RTK data.

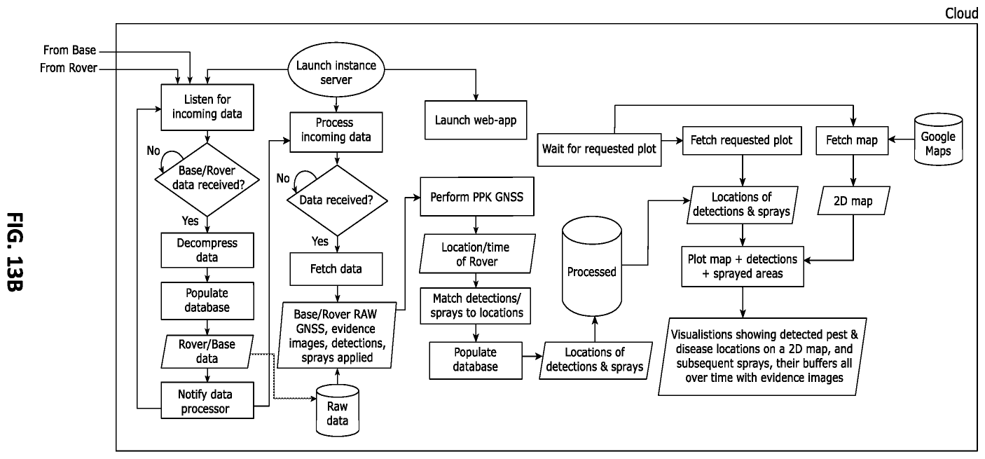

FIG. 14 shows the use case where a user physically mounts and electrically connects the image capture and spraying system.

FIG. 15 shows the use case where a user configures the image capture and spraying system. The user must take rough measurements from each camera to the GNSS antenna to determine the x, y, z location of each camera relative to the GNSS antenna. The same must be done for the computation units and the GNSS antenna. Measurement accuracy of +/−15 cm may be sufficient for most applications.

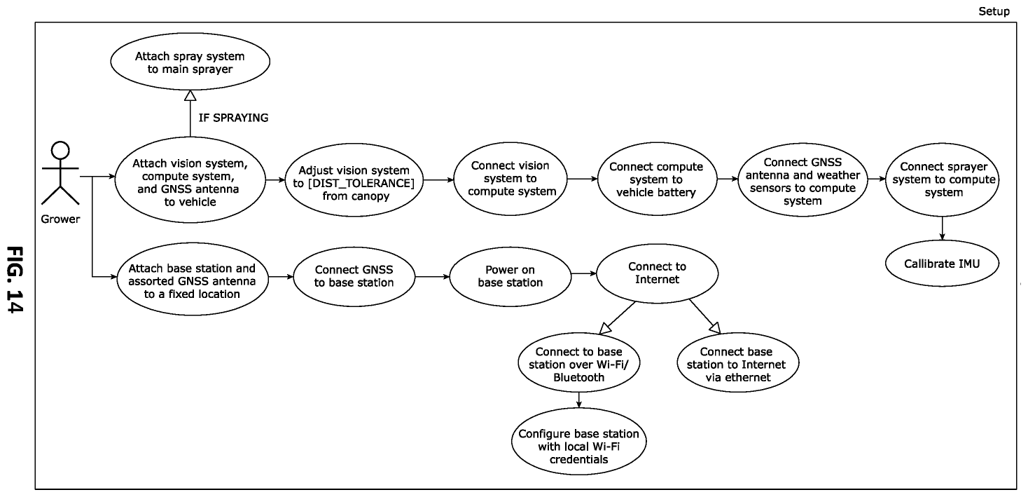

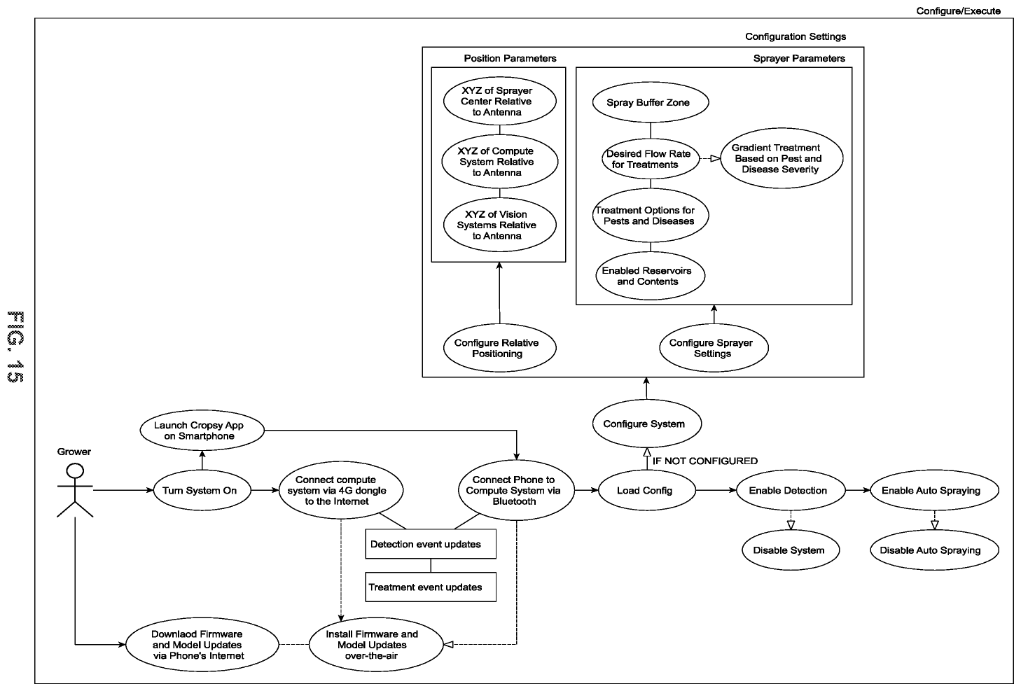

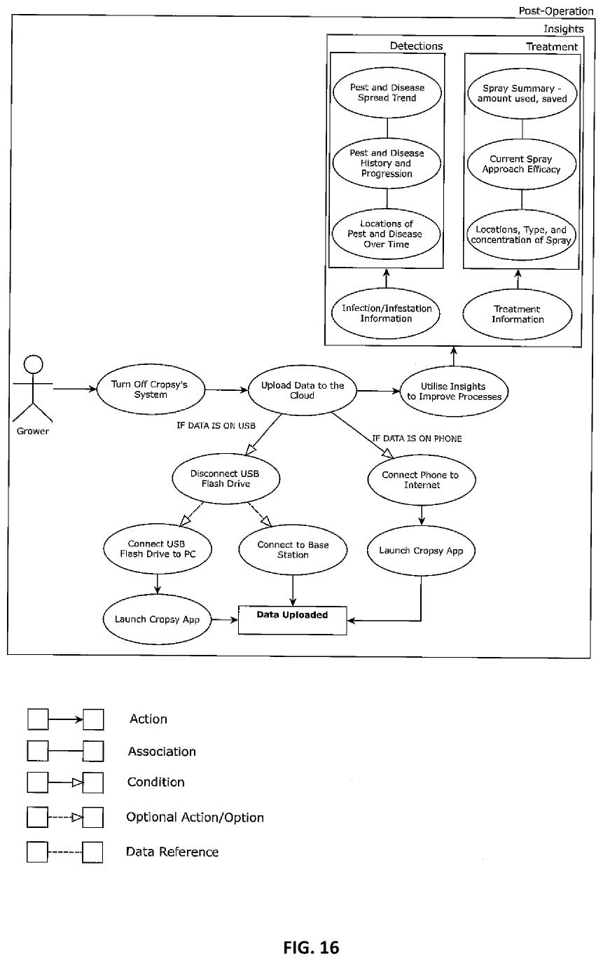

FIG. 16 shows the use case where a user downloads the data to the cloud for post processing, and the insights the user may gain from the data. Insights may include pest and disease occurrence trends over time, pest and disease progression or spread, precise locations of pest and disease occurrence, amount and type of sprays dispensed, current spray plan efficacy, or precise location of sprays dispensed.

In a further embodiment, a user may utilise yield information to optimise the harvesting operations. This includes using the correct number of personnel and harvesters for the amount of produce to be harvested. Also, it includes the positioning optimisations in sending of harvesting personnel and machinery to the correct locations for harvest to minimise unloading time, fuel cost, labour cost, and other cost-related factors.

In a further embodiment, the harvested goods may be optimised using the collected data so that just the right amount of transport vehicles are on-site on a given day to move harvested goods from the harvest personnel and machines to the storage facilities.

In a further embodiment, the storage of harvested fruit may be optimised using the collected data. This may include building extra infrastructure in case of oversupply. Alternatively or additionally this may include:
Booking the correct amount of storage for the expected yield.
Preventing situations where under-supply leads to overpaying for storage.
Over-supply leading to incurred cost for over-storage.

In a further embodiment, the delivery of enough fruit or processed fruit products to satisfy contractual obligations with produce buyers (e.g. wineries, produce distributors, food manufacturers) may be optimised using the collected data. In the case of under-supply, more fruit can be purchased in advance to address under-supply, or fruit sold in advance to address over-supply and improve harvesting, transport, and storage processes.

In a further embodiment, summer pruning processes may be optimised using the collected data. For example, detecting an over-target fruit count results in a targeted fruit dropping where the fruit is cut in the early stages to prevent densification, leading to delayed ripening, increased pests and disease pressure, and others. Another example is shoot-thinning, where excessive shoots are removed to avoid densification of the canopy with fruit. Densification leads to delayed ripening, increased pests and disease pressure, and others.

In a further embodiment, the crop's harvest order may be optimised using the collected data so that only perfectly ripe fruit is picked—not too early and not too late. This is achieved by measuring each plant's and fruit's growth stages, subsequently harvesting suitable areas just-in-time.

In a further embodiment, the placement of bird nets to the areas that have reached adequate ripeness may be determined using the collected data. This is achieved by measuring each plant's and fruit's growth stages, placing bird nets or bird deterrents, on sites that passed a suitable growth stage for the respective crop.

In a further embodiment, winter pruning processes may be optimised using the collected data. For example, detecting an over-target bud count results in a targeted pruning approach where canes or spurs are shortened to reduce the number of buds on them. This in-term leads to fewer shoots growing during the season and results in greater control over quality and quantity of fruit. In another example, counting the number of canes or branches on plants can tell growers if the plants have been pruned to the correct specification (e.g. in vineyards, some blocks require a vine to have three canes and others might require four canes). If plants have not been pruned correctly, the pruning workforce may be retrained or paid according to how many pruning cuts were actually made.

In a further embodiment, measured plant pruning related attributes such as a trunk's, cane's, shoot's, bud's, cordon's size, location, or count, or other attributes relating to the trellis such as wire, tie down straps, zip-ties, nail, post, or others may be used to evaluate pruning quality. For example, in cane pruned canopies, a healthy thick cane that is correctly tied around a wire is considered a good quality prune, whereas a sickly cane that is untied, or broken is considered a bad quality prune, with many variations in between and with different attributes affected. In another example, in spur pruned canopies, a cordon that is correctly pruned where no old spurs are present is considered of good quality, whereas a cordon with old spurs present and uncut is considered of bad quality.

In a further embodiment, plant pruning attributes measured before and after pruning may be used to evaluate pruning quality. For example, in cane pruned canopies, where a four-cane specification is required for the pruning job, a vine may show ten good new-cane candidates prior to pruning, and only two after pruning, indicating a poor quality pruning job where the worker over-pruned. A contrasting example in a pruned canopy with a four cane specification, a plant may only show one good new-cane candidate prior to pruning, and one after pruning, indicating a good quality pruning job. Alternatively, damage to canes may have occurred by machines and not the workers. In a further embodiment, dying, dead, or missing plants may be detected using the collected data. Finding the precise location of dead, dying, or missing plants means these plants can be replaced to maximise utility of the land and increase produce outputs. Moreover, further analysis of the other measured attributes for plants may reveal certain facts about the causes that lead to the missing, dead, or dying plant, for example, the presence of a certain long-term virus/bacteria/fungus or other foreign invasive organism, the use of a non-suitable/underperforming rootstock for the growing condition or land, or other factors.

In a further embodiment, year to year yield information including bud counts, shoot counts, bunch counts, berry counts, and growth stage estimation may be compiled to optimise fertiliser programs and water irrigation processes to improve control over quality and quantity of fruit.

In a further embodiment, year-year yield information including bud counts, shoot counts, bunch counts, berry counts, growth stage estimation, pest and disease detection, may be compiled to optimise spray programs and improve control over quality and quantity of fruit.

In a further embodiment, year to year yield information including bud counts, shoot counts, bunch counts, berry counts, growth stage estimation, pest and disease detection, may be compiled to optimise spray programs to improve scenario planning and risk mitigation.

In a further embodiment, year to year yield and pruning information including trunk's, cane's, shoot's, bud's, cordon's, bunch's, fruit's or other phenological trait's size, location, or count; or other attributes relating to the trellis such as wires, tie down straps, zip-ties, nails, posts; or growth stage estimation; or pest and disease incidence or severity may be processed to prove cause and effect relationships and provide recommendation to growers to maximise yield output and crop quality. For example, in a cane pruned vineyard with two and three cane vines with similar variety and rootstock in a similar area, the three cane vines may show a 10% greater yield than their neighbouring two cane vines. Thus, a recommendation to adjust the pruning strategy for all vines to be three cane is provided to the grower to maximise yield. In another example, vines that are historically infected with a fungus and sprayed with a treatment may show a reduction in yield by 15%, fruit size by 10%, and quality by 50%. Further analysis may show that the reason for the infection is the incorrect spraying of treatment chemicals at the wrong phenological stage, of which a recommendation is made to the grower to spray at the correct phenological stage and minimise yield and quality losses.

In a further embodiment, current season data on pest and disease location and severity may be used to allocate and direct personnel to drop fruit which is unsuitable for harvest before harvest machinery is used.

In a further embodiment, the RTK or PPK location of the device mounted to a vehicle as it moves through its daily operations or on an ad-hoc basis for scanning or other purposes, may be processed to create a 3D topological map of the crop's land. This 3D topological map may be used for crop planning purposes, such as optimising fertilisation or irrigation. For example, for fertilisation, optimisation may occur through the application of variable rate fertilisation based on the land elevation or slope to ensure homogenous delivery of nutrients to all plants across the crop. In another example, for irrigation, water or solution flow rate and volume for lower lying areas may be reduced to ensure a homogenous delivery of water to all plants across the crop.

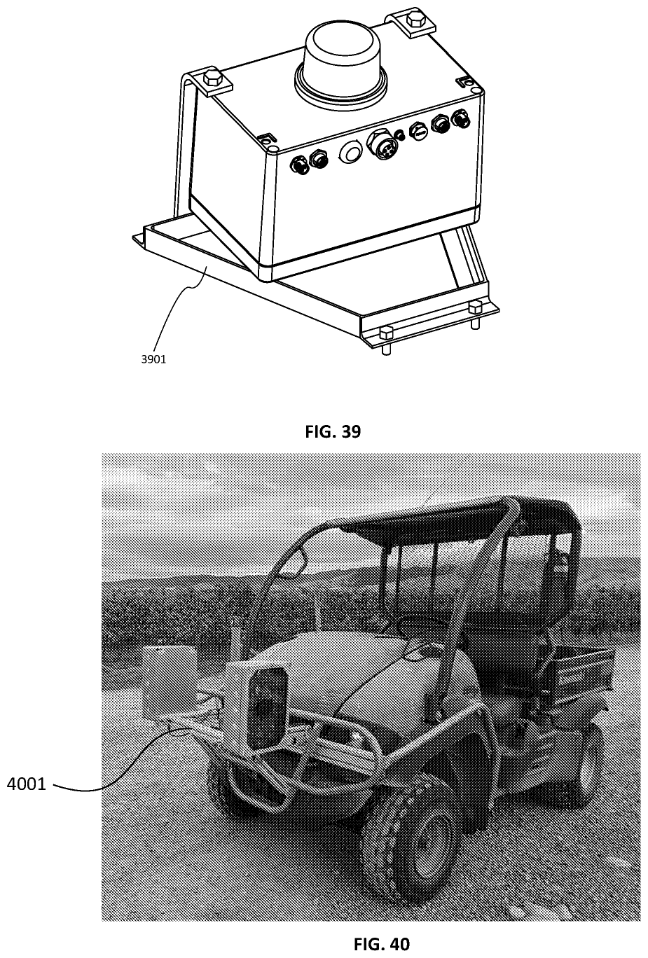

Separate to the image capture system, the controllers may be mounted separately, such as on the roof, front, back, tucked on the side or inside the cabin of the vehicle as to be ‘out of the way’. This is to ensure minimal interference on regular crop operations. The controller may be housed in a waterproof IP65 enclosure. This enclosure may be made of metals such as aluminium or steel or may be made of plastic. The controller may be mounted by being bolted down using a metal sheet frame 3901 as shown in FIG. 39. Alternatively the controller may be mounted using a quick-release latch system, fastened down using ratchet straps, rope or cable ties, or may be placed loosely in a compartment of the vehicle where it will not move around significantly. Alternatively the controller may be attached to frames or mounting systems of the image capture system.

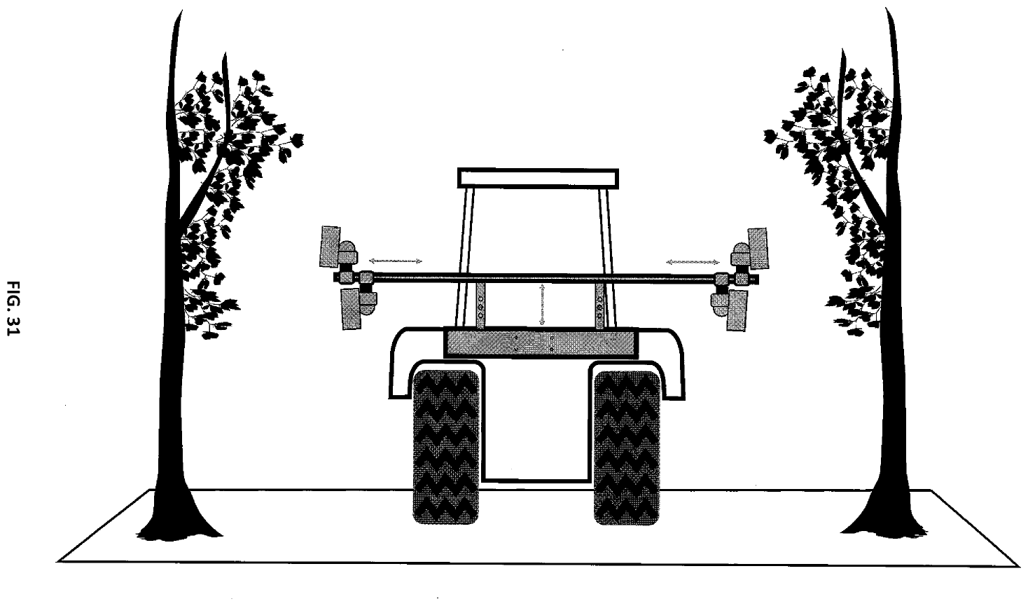

In a further embodiment of the whole system, the image capture devices (including CMOS sensors, over-driven LED package, cross polarisation of the light etc). and the controller system (including the controller, an IMU, GNSS or GPS, LED flash circuit, CMOS sensors driver, blower controller, power supply and spray controller) may be integrated into one package or housing as shown in FIG. 31. The image capture system may still be adjustable through a telescoping mounting system, through suction cups, through a swing arm, or the image capture system may be fixed. The integrated capture system and controller would only require one main attachment to a vehicle rather than attaching the image capture systems and controllers to the vehicle separately. This may be done on a tow bar or front grill of an ATV, or the front weight frame, or rear mount of a tractor.

### Example: Application to Horizontally Trained Crops

Some fruit crops may be trained to grow in a horizontal fashion with the canopy being elevated from the ground, where the canes from plants in two adjacent rows meet in the centre, such that a vehicle may only be able to drive under the canopy, and not have access to the top of the canopy. Some fruit crops grown in this manner may be vine crops, such as grapes.

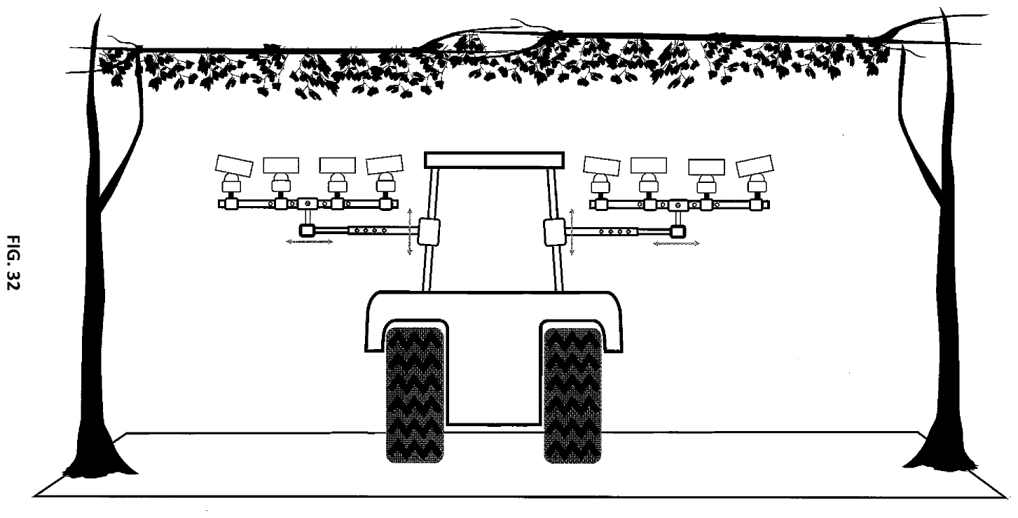

FIGS. 32 to 35 illustrate a vehicle mounted image capture and analysis system 100 according to an example embodiment for horizontally grown thin canopy plants. Any description of components that are common to previous embodiments are also relevant to this example embodiment.

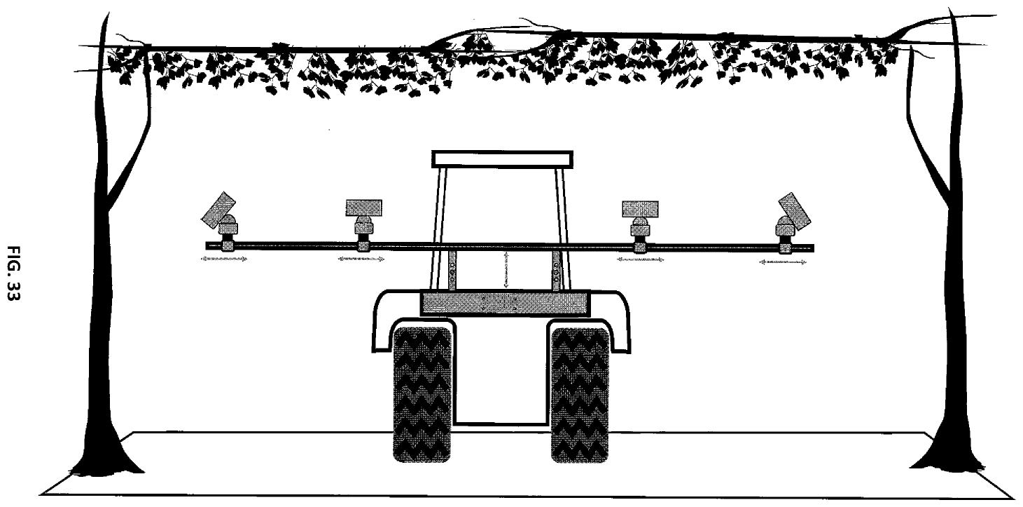

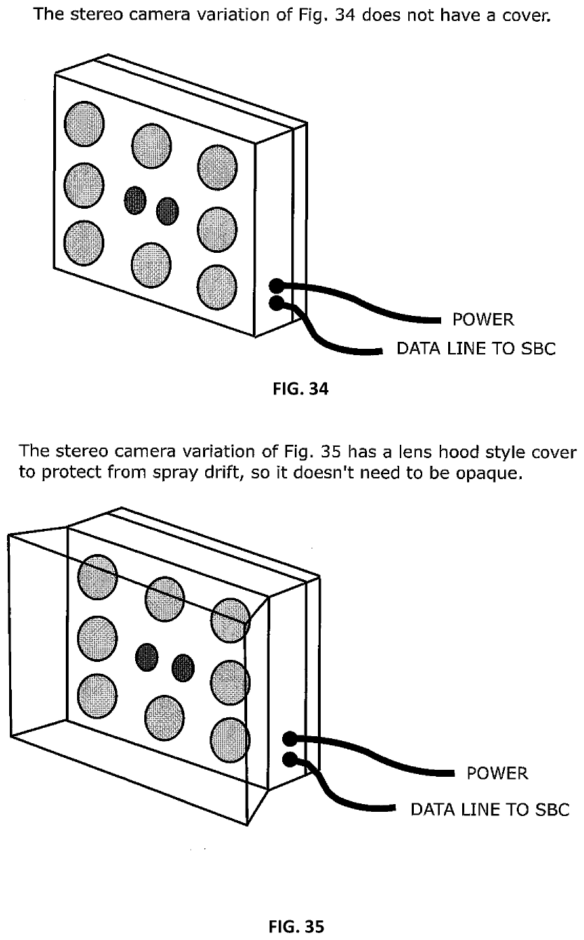

The main features are:
The cameras face up, so that the underside of the canopy may be imaged. The downwards hanging fruit may protrude below the foliage, especially once more mature, and can therefore be easily imaged from underneath. For those fruits that are covered by foliage, or to image the other side of the foliage, the blower described earlier can be used.
The cameras may also face sideways to capture images of the plant trunks, grafts, leaders, or canes. This information maybe be used for identifying, counting, or geo-locating plants for the purposes of tracking how each plant changes over time; viewing girdling points on the plants for the purposes of evaluating the health of the plant and tracking if plants needs to be girdled; identifying or counting canes for the purposes of pruning or other plant management; identifying the age or growth rate of the plants for the purposes of monitoring plant performance over time; identifying suckers on the plant for the purposes of pruning and trimming the plants.
The image data includes depth. The depth can be used to more accurately estimate fruit diameter, volume, weight or other more accurate yield estimates. Even a small increase in accuracy in berry weight estimation can provide large improvements in analytics and eventually to profitability.
The depth data may be provided by Stereo cameras. They may be located side by side as shown in FIGS. 34 and 35. Each image capture module may include 2 CMOS sensors spaced from each other. For example the spacing may be up to 300 mm.
The image capture module may be attached to an autonomous vehicle. The Autonomous vehicle may be purpose designed for specific fruit orchards. The Autonomous vehicle may include functionality specific to certain fruit orchards such as scanning, picking, pruning canes, thinning the canopy, thinning fruits or flowers, or other operations.
An integrated capture system and controller would only require one main attachment to a vehicle rather than attaching the image capture systems and controllers to the vehicle separately.

While the present invention has been illustrated by the description of the embodiments thereof, and while the embodiments have been described in detail, it is not the intention of the Applicant to restrict or in any way limit the scope of the appended claims to such detail. Additional advantages and modifications will readily appear to those skilled in the art. Therefore, the invention in its broader aspects is not limited to the specific details, representative apparatus and method, and illustrative examples shown and described. Accordingly, departures may be made from such details without departure from the spirit or scope of the Applicant's general inventive concept.

1. A method comprising:
activating an illumination source to produce illuminating light;
polarising the illuminating light in a first polarisation axis;
illuminating at least part of a thin canopy plant crop with the polarised illuminating light to produce reflected light;
polarising the reflected light in a second polarisation axis transverse to the first polarisation axis to produce cross-polarised reflected light;
capturing an image of at least part of the thin canopy plant crop using the cross-polarised reflected light; and
analysing the captured image to determine a condition of the thin canopy plant crop.

2. The method of claim 1, wherein the thin canopy plant crop is a vineyard or orchard crop.

3. The method of claim 1 wherein the illumination source and/or the image capture is generally directed upward to the underside of the thin canopy plant crop.

4. The method of claim 1, wherein the capturing an image comprises capturing stereo images.

5. The method of claim 4, wherein the condition of the thin canopy plant crop comprises yield, and yield is determined from the stereo imaging to estimating berry and/or bunch volume/weight.

6. The method of claim 1, wherein the condition of the thin canopy plant crop is selected from the group consisting of:
yield;
pests or disease;
growth stage or maturity of the plant or its fruits;
nutritional deficiencies, for example, nitrogen, phosphorus, potassium, magnesium, boron, zinc;
locating and counting plant suckers;
locating missing, dying, or dead plants;
locating and counting healthy and limp or damaged shoots;
canopy density and leaf-area index;
untreated pruning cuts;
over or under watered plants, and
any combination thereof.

7. The method of claim 1, wherein the condition of the thin canopy plant crop is also determined using input from one or more of: temperature sensors, relative humidity sensors, soil moisture sensors, barometric pressure sensors, wind sensors, UV sensors, light sensors, depth sensors and/or rain sensors.

8. The method of claim 5, wherein yield is determined either directly, or indirectly, where directly comprises imaging and counting fruits, berries, bunches, or buds, blossoms, inflorescences which will later turn into fruits, or imaging and counting fruits unsuitable for harvest due to pest, disease, or other damage; where indirectly comprises estimating yield from counting shoots, identifying the growth stage of the plant or fruits over time to forecast how much of the crop will reach maturity, depth imaging and estimating berry and/or bunch volume/weight, and using pest and disease information to forecast how much of the crop may be affected.

9. The method of claim 1, wherein the cross-polarised reflected light has a greater intensity than polarised ambient light that has reflected from the plant and been captured along with the image

10. (canceled)

11. The method of claim 9, wherein the intensity of the cross-polarised reflected light is more than 10 times greater than the intensity of the reflected ambient light.

12. The method of claim 1, wherein analysing the captured image comprises performing surface analysis of the imaged part of the thin canopy plant crop.

13. The method of claim 12, wherein the surface analysis comprises segmenting the image based on visual symptoms of the condition detected on the surface of the thin canopy plant crop, or features of the thin canopy plant crop.

14. The method of claim 1, wherein the first polarisation axis is at approximately 90° to the second polarisation axis.

15. The method of claim 1, wherein the illumination source is pulsed or strobed.

16. An image capture system comprising:
an illumination source configured to produce illuminating light to illuminate a thin canopy plant crop;
a first polariser arranged to polarise the illuminating light from the active illumination source;
a second polariser arranged to cross-polarised light reflected from the thin canopy plant crop, the second polariser having a polarisation axis transverse to the polarisation axis of the first polariser; and
an image capture device configured to capture light cross-polarised by the second polariser;
wherein the active illumination source is configured to produce illuminating light of sufficient intensity that the light cross-polarised by the second polariser is of greater intensity than ambient light reflected from the thin canopy plant crop and polarised by the second polariser.

17. The image capture system of claim 16, wherein the image capture system is integrated with a controller and the integrated components have a single mounting system to a vehicle.

18. The image capture system of claim 16, wherein the image capture device is configured to capture a stereo image of the light cross-polarised by the second polariser.

19. (canceled)

20. The image capture system of claim 16, wherein the active illumination source has a total output intensity of at least 1,100,000 lumens.

21. The image capture system of claim 16, wherein the active illumination source comprises one or more light-emitting diodes and the image capture system is configured to strobe the light-emitting diodes with an intermittent voltage above a rated LED voltage.

22. (canceled)

23. A vehicle mounted image capture system, comprising:
a high intensity illumination source configured to strobe foliage or fruit of a target thin canopy plant;
an image capture device configured to capture images of the illuminated foliage and/or fruit;
an on-board computation unit(s) to process images of the illuminated foliage and/or fruit in real-time;
a housing integrating the illumination source, the image capture device and the computation unit; and
a single mounting system for the housing to the vehicle.
